(19) United States
(12) Patent Application Publication
(10) Pub. No.: US 2013/0022562 A1
(43) Pub. Date: Jan. 24, 2013

(54) NOVEL CEREAL PRODUCTS, PRODUCTION AND USE THEREOF, AND COSMETIC FORMULATIONS CONTAINING THEM

(71) Applicants: Cark Maunsell, Southampton (GB); Olavi Myllymäki, Helsinki (FI); Martti Huhtakallio, Jalasjarvi (FI); Ilkka Lehtomäki, Helsinki (FI)

(72) Inventors: Cark Maunsell, Southampton (GB); Olavi Myllymäki, Helsinki (FI); Martti Huhtakallio, Jalasjarvi (FI); Ilkka Lehtomäki, Helsinki (FI)

(73) Assignee: Oat Services Ltd., Southampton (GB)

(21) Appl. No.: 13/550,710

(22) Filed: Jul. 17, 2012

Related U.S. Application Data
(60) Provisional application No. 61/508,706, filed on Jul. 18, 2011.

(30) Foreign Application Priority Data
Jul. 18, 2011 (FI) 20115758

(51) Int. Cl. A23L 1/10; A61K 8/97; C09D 197/02; A61Q 19/02; A61Q 19/08; A61Q 19/00; B02B 5/02; A61Q 17/04

(52) U.S. Cl. 424/59; 426/622; 241/25; 424/725; 424/62; 106/137.1

(57) ABSTRACT

This invention relates to novel cereal products, especially natural cereal products of colloidal nature. The invention also relates production and uses of colloidal cereal products. Further the invention relates to cosmetic formulations containing cereal products.

### CROSS REFERENCE TO RELATED APPLICATIONS

Applicants hereby claim foreign priority benefits under U.S.C. §119 from Finnish Patent Application No. 20115758 filed on Jul. 18, 2011 and from U.S. Provisional Patent Application Ser. No. 61/508,706 filed Jul. 18, 2011, the contents of both which are incorporated by reference herein.

### FIELD OF THE INVENTION

This invention relates to novel cereal products having enhanced colloidal properties. Particularly this invention relates to cereal products that are suitable for use in cosmetic and food applications. Further this invention relates to a method of producing them, their use in food and cosmetic formulations containing said cereal products.

### BACKGROUND

Cereal flours especially oat (Avena sativa) flour has been used for over a 1,000 years as an ingredient in cosmetic products to protect and repair the skin. Oat flour contains components that have anti-itching and anti-inflammatory properties. (Pomeranz 1995). Oats also contain natural antioxidants and compounds capable of absorbing UV-light (Kurtz and Wallo 2007). In Finland and the UK oats, in contrast to wheat, barley and rye, are permitted in celiac diets. Oat starch grains are similar in size (approximately 8 microns) to rice and smaller than other cereal starches. These properties make oat flour an ideal component for cosmetic products. Oat flour has been used for example in moisturising creams, anti-aging products and for use with sensitive skins for a long period. One example of an oat ingredient that is used and that has a defined specification for cosmetic industry is colloidal oatmeal (Kurtz and Wallo 2007). The colloidal oatmeal specification is described in the United States Pharmacopeial Convention USP32-NF27 page 2024, where the following characteristics are prescribed: Maximum dry matter, nitrogen, fat, protein, ash content, microbial assays, particle size and viscosity. The properties and allowable claims are described in the Federal Drug Administration (FDA) Federal Register Vol. 69, No. 160, 19 Aug. 2004.

Cereal flours contain a starch fraction that is easily gelatinized during heating with moisture. Gelatinisation is enhanced by mechanical energy, for example by shearing. Gelatinisation increases the viscosity of the flour in wet conditions and also modifies other properties such as the size of starch particles. It is advantageous that the level of gelatinisation is controlled for different applications, for example, cosmetics or food beverages. This invention controls the gelatinisation during extrusion.

Cereal flours may naturally have high a microbial content, which may be controlled or reduced during processing. Other typical effective control methods such as the use of chemicals or irradiation are not accepted by many cosmetic manufacturers. This invention significantly improves the microbial purity during natural extrusion and is an allowable practice by most cosmetic manufacturers.

Typical methods of controlling microbial purity are:

Beta or Gamma Irradiation, which produces ions that improve the microbial purity of cosmetic products. The process is limited to small finished packs or paper containers but larger unit volumes can be more problematic as the surface may become brownish and the middle of the bag may not be correctly irradiated. Irradiation can cause physicochemical changes, which are not beneficial when using the ingredient as a cosmetic product. Retail consumers may also be concerned with the use of irradiated products and the cost of this process tends to be high. The maximum allowed irradiation in the food industry is 10 kGy (kilogray) and the normal dosage in cosmetic ingredients is 2-10 kGy. Starch requires higher loads of over 5 kGy, as it is hygroscopic and may protect contaminants.

Chemical disinfection is an alternative method, but the chemicals may bring with them challenges such as increased allergenic activity as well as potentially altering the flour structure. These types of molecules may be prohibited by some cosmetic manufacturers.

Hydrothermal methods usually require high moisture and high temperature. Typical examples are pasteurisation, UHT (Ultra-high temperature treatment) and autoclaving. The parameters for these treatments may lead to significant changes in the behaviour of the flour due to gelatinisation of the starch fraction. Extrusion is also regarded as a hydrothermal method, but involves a solid material, where the moisture is low when compared to the other hydrothermal methods.

In an extruder moistened flour is rapidly modified when it is pressurised by the force exerted by its passage along an Archimedes screw within the barrel at the end of which pressure can be further increased through the use of nozzles. An alternative to this is the use of an expander where the nozzles are replaced by hydraulic backpressure. The barrel may consist of a number of cylinders that can be either heated or cooled. The number of cylinders defines the length of barrel and the associated shaft and screws. An extruder has usually three sections described as the feeding, mixing and reaction chambers.

The extruder settings cannot be changed when the extruder is in operation. The extruder settings are for example, the different pitches and the shapes of the screws in the feeding, mixing and reaction sections, special tools in the mixing section and different nozzle settings. One of the significant settings is the use of the reverse screw, which is located in front of the nozzles to create back-pressure. Nozzles are located on a nozzle plate, and can be off different shapes and sizes. The extruder settings will usually vary according the raw material and objective of the extrusion.

The operation settings such as feed rate, speed of screw rotation, moisture and temperature can be adjusted during processing.

During extrusion normal flour moisture levels are between 15 and 35 w %. Full starch gelatinisation requires 61 w % water and 39 w % starch (Wang 1993). Therefore in traditional flour extrusion the moisture level is not sufficient for full starch gelatinisation even though the starch level of the flour is 60-70 w %. This significant phenomenon gives a means to control the degree of gelatinisation during extrusion.

Flour Characteristics:

Cereal flours consist of several components e.g. water, protein, carbohydrates (starch), fat and ash. The relative level of these components varies in different flours. These variations have a significant effect on the extrusion process. In conventional extrusions the degree of starch gelatinisation is not controlled in addition to which other components are changed. These types of extruded flours would not be the most suitable for use in cosmetics or for incorporation in certain food applications.

Oil in flour tends to create a slip layer on the surface of the starch particles during extrusion. The addition of 0.5-1.0 w % of vegetable oil decreases the mechanical energy in extrusion cooking when other extrusion variables are kept constant. The role of oils as lubricants can be observed in the low moisture extrusion of starches that have low lipid content such as potato starch, which can overheat at the contact surfaces causing problems in starch transport through the barrel and possibly blockages due to the creation of degraded material. Increasing the oil content up to 2-3% markedly prevents the starch dispersion, which decreases the melt viscosity. This significant phenomenon leads to higher levels of uncooked dough (Ilo et al. 2000).

Oats have higher levels of oil dispersed throughout the kernel in contrast to other cereals such as wheat, maize, rice, barley and rye, which have the majority of the oil in the germ. The germs can be easily mechanically fractioned out of the kernels during the milling process. The level of oil in oat flour is 2-3 times higher than other cereal flours (Ilo et al. 2000).

The extruder mechanical energy has a significant effect on the degree of gelatinisation. The level of extruder mechanical energy is affected by the following parameters:

1. The characteristics of the raw material

2. Extruder settings
Length of the screw
Screw setting
Nozzle setting

3. Operation settings
The feed rate
The moisture
The relative temperature in parts of the barrel
The rotational speed of the screw

The level of mechanical energy can be assessed by measuring the pressure at the entry point to the extruder nozzles. Similarly the pressure is affected by the same parameters as mechanical energy. The pressure in conventional extrusions is in the range 50-150 bar.

Combined Effect of Extruder Settings and Flour Characteristics:

It is known that the water activity is an important factor in decreasing microbial content with the use of heat. The addition of oil decreases the water activity. In an extruder the screws and especially the reverse screw create a plasticised mass, where the moist flour with added oil is very efficiently mixed whilst heated. This highly efficient mixing effectively reduces microbe count.

One drawback in the prior art solutions is that conventional extruder settings together with operational settings will create flours where the starch fraction and other flour components have been excessively modified e.g. the starch is over-gelatinised and lipids deteriorated in quality. The quality of resultant flour is not ideal for cosmetic use. Furthermore, current cosmetic flour ingredients seldom fulfil the natural cosmetic requirements such as those defined under the Ecocert Natural certification scheme or the European standard, COSMOS, where the use of irradiation, disinfection chemicals and the current hydro-thermal treatments are not permitted treatments.

There is a need to obtain a high quality cereal product with the characteristics of colloidal oatmeal, which does not compromise the microbial quality and does not utilise unapproved chemical treatments or irradiation to do so. Furthermore, there is a need for cereal products, which have improved oil retention and increased viscosity for use in cosmetic, food, and coating applications.

### SUMMARY

The object of the current method is to naturally modify the flour in order to reach controlled and minimal levels of gelatinisation resulting in improved characteristics in cosmetics and foods. This invention provides specific cereal products for use in food and cosmetic application and a method for their production. During processing raw materials, the microbial purity of the flour is naturally improved and the behaviour modified in a controlled manner resulting in flour which is adapted for cosmetic use. In addition the method according to the invention ensures that the endogenous enzymes within the raw material, specifically oats, are effectively neutralised. This enhances the shelf life of the resultant ingredient and in the corresponding cosmetic product.

It is already known that an extruder improves the microbial purity of the flours (Cheftel 1998), and in addition to that, an extruder, expander or a similar heating screw is a usual means to control the pathogens e.g. salmonella, in animal feeds. However these methods ignore major changes to flour structure and starch gelatinisation since the nutritional content is the important factor in animal feed. This invention presents an extruded flour product, where the microbial purity has been significantly improved and the behaviour of the flour is controllably changed. Thus the flour is therefore adapted for cosmetic and food use.

This invention also describes the effect of extrusion on the enforced colloidal behaviour, improving flour solubility and phytochemical (betaglucan, avenanthramides, and protein) availability.

These and other objects are achieved by the present invention as hereinafter described and claimed.

The first aspect of the invention is a cereal product. The cereal product is mainly characterized by that stated in the descriptive part of claim 1.

The second aspect of this invention is a method for producing a cereal product. According to this invention the cereal raw material is extruded and finely milled. More specifically the method according to the present invention is mainly characterized by the description outlined in claim 9.

The third and fourth aspects of this invention are the uses of the cereals products described here in food or cosmetic products or as a coating barrier in protective products.

The fifth aspect of the invention is a cosmetic formulation. The characteristic to said formulation is that it contains cereal product(s) described here.

The preferred embodiments of the invention are disclosed in the dependent claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fees.

FIG. 1. The figure shows curves of viscosity versus shear rate of the extruded and non-extruded colloidal oatmeal (dry matter at 10%).

FIG. 2. The photograph demonstrates the difference between the centrifuged colloidal oatmeal (dry matter 5 w %). The left test-tube is the control containing oatmeal that is conventionally milled and the right test-tube contains oatmeal that has been extruded and milled.

FIG. 3. The photographs (magnification 400×) show the iodine colourised colloidal oatmeal particles. The extruded version is on the left-hand side and the conventional flour on the right-hand side.

### DESCRIPTION OF PREFERRED EMBODIMENTS

Colloidal oatmeal is specified in the United States Pharmacopeial Convention USP Monograph USP32-NF27 page 2024, which specifically includes flour particle size. Whole grain oat flour together with the use of the extrusion process meets the specified characteristics for colloidal oatmeal, with the exception of particle size. Therefore the extruded and dried whole grain oat flour must be further milled to meet this requirement. The colloidal behaviour of flours requires the use of very small particle sizes in order for any cereal flour to achieve a colloidal state.

The extruded cereal flour product of this invention has the following characteristics:
The flour characteristics have been changed during extrusion in a controlled manner and the changes are slight in order to give flours adapted for cosmetic and food use.
The extruded flour can be regarded natural or certified under Ecocert or COSMOS as ‘natural’.
The extruded flour can replace flour products, where in order to attain microbial purity unapproved methods such as irradiation have been used.

In one embodiment a cereal product which has of at least 0.4%, preferably at least 0.6%, more preferably 0.8%, even more preferably 1.0% and most preferably at least 1.2% dissolved material as measured using the Brix method from 10 w-% water suspension. In this connection the Brix value indicates the approximate value of dissolved solids. The dissolved components include starch as well as beta-glucan, avenanthramides and flavonoids in a bioavailable form and the claimed product is thus improved compared to conventional colloidal flours.

In one embodiment a cereal product is natural or capable of being certified by the certifying bodies, Ecocert or COSMOS as natural. In this connection “natural” means compliant with the COSMOS cosmetic and natural standards (Currently Version 1.1 dated 31 Jan. 2011), excluding processes such as ionizing radiation (Appendix III), and only approved ingredients of mineral origin (Appendix IV), or other permitted ingredients (Appendix V). There is increasing demand for certified natural products within both cosmetic and food applications.

In one embodiment a cereal product has total microbial count less than 5000 cfu/g (measured from the sample as-is), preferably less than 3000 cfu/g, more preferably less than 1000 cfu/g, even more preferably less than 500 cfu/g, even more preferably less than 100 cfu/g, and most preferably less than 10 cfu/g. Microbial purity/quality is an essential feature in cosmetic and food applications.

In one embodiment the endogenous enzymes, specifically lipase, of the cereal product are destroyed in the extrusion process. Active cereal enzymes have an adverse effect on to the quality of the product and shelf life.

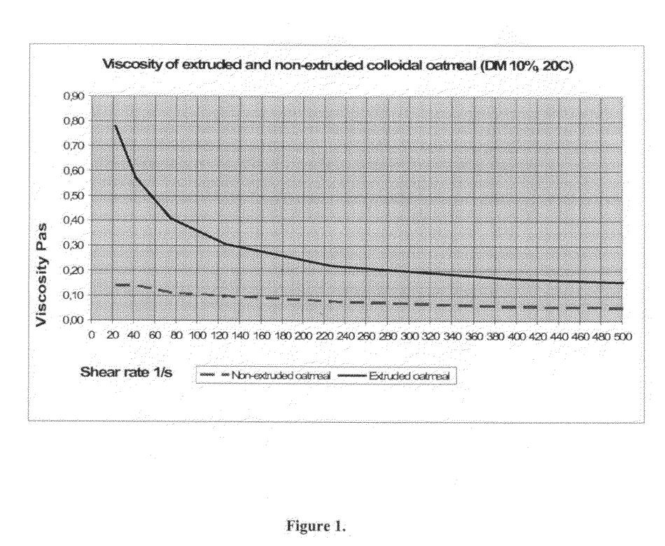

In one embodiment a cereal product has viscosity of at least 0.150 Pas, preferably at least 0.170 Pas measured at 20° C. from 10% water suspension using a shear rate in the range of 20-40 1/s, preferably 22 to 25 Ws, which is the lowest shear rate in the FIG. 1. Enforced viscosity ensures that the product forms effective creams and lotions in cosmetic applications and is also suitable for food applications. Furthermore, such a product has an increased amount of free beta glucan, which is known to be an excellent film former as well as has the ability to penetrate the skin. (Pillai et al 2005).

In one embodiment a cereal product is of colloidal nature. The United States Pharmacopeial Convention for colloidal oatmeal also defines the maximum viscosity for colloidal oatmeal and the corresponding method of assay. The viscosity of 4.5% colloidal oatmeal must be less than 0.1 Pas. The method of measuring viscosity according to the monograph is described in the examples. In one embodiment of the invention the viscosity of 4.5% colloidal cereal product is be less than 0.1 Pas.

In one embodiment a cereal product has improved oil retention properties compared to non-extruded flour product. One reason for this is that extrusion releases amylose, which complexes and binds lipids (FIG. 3). This phenomenon is known to extend the shelf life of the products. The increased oil content gives improved skin feel in pressed powders e.g. eye shadow, as well as improving the moisturising properties of the oatmeal.

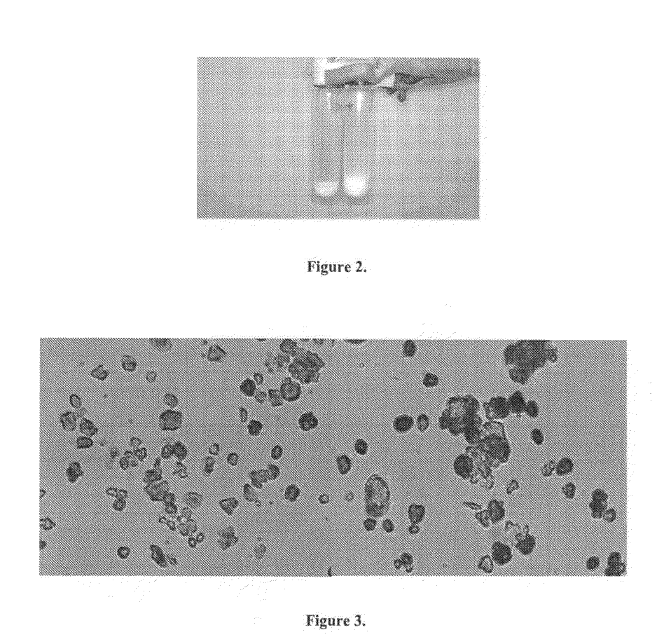

In one embodiment a cereal product has reduced and modified particles, which improves the colloidal nature of the flour as well as improving skin feel. (FIG. 2)

In one embodiment the starch of a cereal product is slightly gelatinized in a controlled manner, whereas material made from non-extruded flour was not gelatinized. Partial gelatinization improves the solubility and maintains the colloidal nature of the product. Partial gelatinization also partially releases amylose.

In a preferred embodiment of the product the constituent cereal is oats. Oats have increased levels of an endogenous polysaccharide, beta glucan. Beta glucan is known to have immunestimulent, moisturising and film forming properties. Small starch grains of oats, when compared to wheat, maize, barley and rye, are well adapted for cosmetic applications. The nutritional benefits of beta glucan have previously been discussed in detail e.g. in WO 2004/099257.

Oats also have a high oil content (compared to wheat, maize, barley and rye), which brings high levels of natural antioxidants and emulsifiers, thereby improving moisturisation and skin feel.

The extrusion method described here includes the extruder settings and the operational parameters, which together with the correct specification of raw material flour characteristics are suitable for producing colloidal oatmeal.

One embodiment of the invention is a method for producing a cereal product that comprises the steps of extruding the cereal flour and fine grinding of the extruded flour. According to the invention the conditions in extrusion chamber are controlled by
(a) maintaining the moisture below 17 w %, preferably below 15 w % and most preferably below 13 w %; or
(b) optionally adding oil(s) into the feed at 1 to 6 w %, preferably 2 to 4 w % and most preferably about 3 w %; or
(c) lowering pressure of extrusion chamber by 20%, preferably 40% and even 60% compared to conventional pressure in chamber; or
(d) any combination of steps (a) to (c).

Usually the oil content in oats is suitable for the process, whereas other cereals have lower endogenous fat contents and thus require the addition of oil, typically vegetable oil. The oil component, which is added during extrusion, must be capable of sustaining temperatures of 120° C. without suffering any deterioration of quality during extrusion. If the oil concentration on entry to the extruder or expander exceeds 14 w % part of the oil may separate as a free phase.

One embodiment of the invention is a method that comprises the steps of extruding the cereal grain and fine grinding, wherein the pressure of the extrusion device is lowered under controlled conditions. The pressure of the extrusion chamber can be lowered by mechanical means e.g. widening the nozzles or removing the nozzle plate of the extruder. In a preferred embodiment the effective (back) pressure is created by the use of a reverse screw, with enlarged nozzles or without a nozzle plate.

The extrusion process efficiently kills the unwanted microbes in the feed without the need of additional treatments e.g. by irradiation or chemicals or the use of other hydrothermal methods such as pasteurization, UHT-treatment or autoclaving. Hydrothermal methods require large quantities of water resulting in uncontrolled gelatinization of starch. Irradiation or chemical treatments are not preferred treatment by some consumers. In this process the moisture during extrusion should be low in order to avoid over-gelatinization of the product.

Usually dehulled oats are stabilized by heat-treating whole dehulled kernels in order to destroy the endogenous enzyme, lipase. In the extrusion process described here wholemeal oat flour can be stabilized. This ensures that endogenous lipase and other enzymes located also in the core of the kernel are destroyed. A separate stabilization step is not therefore required which increases process economy.

Another benefit of the method of this invention is the partial release of amylose, which results in improved oil binding, enforced colloidal nature and more suitable viscosity. These benefits have been already discussed above in connection with the cereal product.

In one embodiment of this cereal product invention is that it is used in preparation of a cosmetic product, which term is also intended to include toiletries compositions. The benefits of oats, and particular colloidal oatmeal has been known for some time (Kurtz E S et al 2007), and this invention enhances the properties of colloidal oatmeal by increasing oil binding and the colloidal properties giving an improved skin feel as well as enhanced soothing properties.

In another embodiment of this cereal product invention it is used in preparation of a food product. The role of beta glucan in nutrition and health is discussed in other patents e.g. WO 2004/099257.

In still another embodiment the cereal product of this invention is used as a coating barrier in protective products such as gloves, especially latex or nitrile gloves. The use of colloidal oatmeal as a coating on barrier protective gloves requires the oatmeal to have very low particulate concentration within the lining of the glove, and be capable of showing effective moisturisation, anti-irritancy or redness reduction properties at the defined oatmeal concentration of 0.007 w % [FDA Monograph Vol. 69, No. 160, 2004]. Neuser et al describe the benefits of colloidal oatmeal in U.S. Pat. No. 7,691,436B2. The cereal product according to our invention enhances the solubility of the colloidal oatmeal thereby reducing particulate concentration, and as the solubility of the beta-glucan and other active molecules is improved through the process this enhances the moisturisation, anti-irritancy and redness reduction capabilities.

This invention also concerns cosmetic formulations containing the cereal product described above. The cosmetic formulation has the following specific characteristics:
The colloidal behaviour is enforced by extrusion
The particle behaviour enforces the colloidal property
The presence of beta glucan is maximised during extrusion
The amylose fraction is partially freed during extrusion
The cosmetic ingredient can be certified as natural

In an embodiment the cosmetic formulation contains a cereal product described here, or a product obtained using a method as described here. The cosmetic formulation may be a balm, lotion, mask, shampoo, hair conditioner, soap, moisturizer, sunscreen, peeling cream, powder, without restricting to these.

The cosmetic and/or toiletries compositions (or formulations) of the present invention may contain 0.05 to 50% of the Extruded colloidal oatmeal by weight of the total composition. In compositions intended to be applied topically to the skin to protect the skin the amount of Extruded colloidal oatmeal that may be present is preferably in the range 0.5 to 10%, more preferably 1 to 6%.

The compositions of the present invention may contain a safe and effective amount of one or more inorganic and organic sunscreening agents Suitable inorganic sunscreening agents include:

a) Microfine titanium dioxide;
b) Microfine zinc oxide; and
c) Boron nitride.

Examples of suitable additional organic sunscreening agents include:
a) para-aminobenzoic acids, esters and derivatives thereof, for example, 2-ethylhexyl para-dimethylaminobenzoate and the octyl ester of para-aminobenzoic acid;
b) methoxycinnamate esters such as 2-ethylhexyl para-methoxycinnamate, 2-ethoxyethyl para-methoxycinnamate or α,β-di-(para-methoxycinnamoyl)-α′-(2-ethylhexanoyl)-glycerin;
c) benzophenones such as oxybenzone;
d) dibenzoylmethanes such as 4-(tert-butyl-4′-methoxydibenzoylmethane;
e) 2-phenylbenzimidazole-5 sulfonic acid and its salts;
f) alkyl-β,β-diphenylacrylates for example alkyl α-cyano-β,β-diphenylacrylates such as octocrylene;
g) triazines such as 2,4,6-trianilino-(p-carbo-2-ethyl-hexyl-1-oxi)-1,3,5 triazine; and
h) camphor derivatives such as methylbenzylidene camphor.
i) organic pigments sunscreening agents such as methylene bis-benzotriazxole tetramethyl butylphenol
j) silicone based sunscreening agents such as dimethicodiethyl benzal malonate

The sunscreening agents of the present invention may be incorporated into sunscreen products such as oil phase dispersions or emulsions in the conventional way. The emulsion may be an oil-in-water emulsion:

The oil phase of the oil phase dispersions and the water-in-oil and oil-in-water emulsions of the present invention may comprise for example:
a) hydrocarbon oils such as paraffin or mineral oils;
b) waxes such as beeswax or paraffin wax;
c) natural oils such as sunflower oil, apricot kernel oil, shea butter or jojoba oil;
d) silicone oils such as dimethicone, cyclomethicone or cetyldimethicone;
e) fatty acid esters such as isopropyl palmitate, isopropyl myristate or dioctylmaleate;
k) fatty alcohols such as cetyl alcohol or stearyl alcohol; or
g) mixtures thereof, for example, the blend of waxes available commercially under the trade name Cutina (Henkel).

In preferred water-in-oil compositions of the present invention the oil phase comprises 5 to 40%, more preferably 10 to 30% by weight of the composition. In preferred oil-in-water compositions of the present invention the oil phase comprises 5 to 30%, more preferably 10 to 20% by weight of the composition.

The emulsifiers used may be any emulsifiers known in the art for use in water-in-oil or oil-in-water emulsions. It has been found that particularly effective water-in-oil and oil-in-water sunscreen compositions can be prepared by using an emulsifier or mixture of emulsifiers selected from known cosmetically acceptable emulsifiers which include; sesquioleates such as sorbitan sesquioleate, or polyglyceryl-2-sesquioleate; ethoxylated esters of derivatives of natural oils such as the polyethoxylated ester of hydrogenated castor oil; silicone emulsifiers such as silicone polyols; anionic emulsifiers such as fatty acid soaps e.g. potassium stearate and fatty acid sulphates e.g. sodium cetostearyl sulphate; ethoxylated fatty alcohols; sorbitan esters; ethoxylated sorbitan esters; ethoxylated fatty acid esters such as ethoxylated stearates; ethoxylated mono-, di-, and tri-glycerides; non-ionic self-emulsifying waxes; ethoxylated fatty acids; mixtures thereof.

The compositions of the present invention may additionally comprise other components which will be well known to those skilled in the art. These include, for example, emollients such as isopropyl myristate or triglycerides of fatty acids e.g. lauric triglyceride or capric/caprylic triglyceride, such as the triglyceride available commercially under the trade name Migliol 810 (Huls UK); moisturisers such as D-panthenol; humectants such as glycerin or 1,3-butylene glycol; antioxidants such as DL-α-tocopherylacetate or butylated hydroxytoluene; emulsion stabilising salts such as sodium chloride, sodium citrate or magnesium sulphate; film formers to assist spreading on the surface of the skin such as alkylated polyvinylpyrrolidone e.g. available commercially under the trade name Antaron (GAF); thickeners such as acrylic acid polymers e.g. available commercially under the trade name Carbopol (B.F. Goodrich) or modified celluloses e.g. hydroxyethylcellulose available commercially under the trade name Natrosol (Hercules) or alkylgalactomanans available under the trade name N-Hance; preservatives such as bronopol, sodium dehydroacetate, polyhexamethylenebiguanide hydrochloride, isothiazolone or diazolidinylurea; sequestering agents such as EDTA salts; perfumes and colourings.

The compositions of the present invention may also contain a safe and effective amount of one or more anti-acne actives. Examples of useful anti-acne actives include resorcinol, sulfur, salicylic acid, benzoyl peroxide, erythromycin, zinc, etc.

The compositions of the present invention may further contain a safe and effective amount of one or more anti-wrinkle actives or anti-atrophy actives. Exemplary antiwrinkle/anti-atrophy actives suitable for use in the compositions of the present invention include sulfur-containing D and L amino acids and their derivatives and salts, particularly the N-acetyl derivatives, a preferred example of which is N-acetyl-L-cysteine; thiols, e.g. ethane thiol; hydroxy acids (e.g., alpha-hydroxy acids such as lactic acid and glycolic acid or beta-hydroxy acids such as salicylic acid and salicylic acid derivatives such as the octanoyl derivative), phytic acid, lipoic acid; lysophosphatidic acid, skin peel agents (e.g., phenol and the like), vitamin B compounds and retinoids which enhance the keratinous tissue appearance benefits of the present invention, especially in regulating keratinous tissue condition, e.g., skin condition.

The compositions of the present invention may also contain a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably retinol, retinol esters (e.g., C2-C22 alkyl esters of retinol, including retinyl palmitate, retinyl acetate, retinyl propionate), retinal, and/or retinoic acid (including all-trans retinoic acid and/or 13-cis-retinoic acid), more preferably retinoids other than retinoic acid.

Additional peptides, including but not limited to, di-, tri-, tetra, penta and hexapeptides and derivatives thereof, may be included in the compositions of the present invention in amounts that are safe and effective. As used herein, “peptides” refer to both the naturally occurring peptides and synthesized peptides. Also useful herein are naturally occurring and commercially available compositions that contain peptides.

The compositions of the present invention may include a safe and effective amount of an anti-oxidant/radical scavenger. The anti-oxidant/radical scavenger is especially useful for providing protection against UV radiation which can cause increased scaling or texture changes in the stratum corneum and against other environmental agents which can cause skin damage.

A safe and effective amount of an anti-oxidant/radical scavenger may be added to the compositions of the subject invention, preferably from about 0.1% to about 10%, more preferably from about 1% to about 5%, of the composition.

Anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate. tocopherol acetate, other esters of tocopherol. butylated hydroxy benzoic acids and their salts, 6-hydroxy acid (commercially available under the tradename Trolox®), gallic acid and its alkyl esters, especially propyl galate, uric acid and its salts and alkyl esters, sorbic acid and its salts, esters of tocopherol, lipoic acid, amines (e.g., N,N-diethylhydroxylamine, amino-guanidine), sulfhydryl compounds (e.g., glutathione), dihydroxy fumaric acid and its salts. lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used.

The compositions of the present invention may also contain a safe and effective amount of a chelator or chelating agent. As used herein, “chelator” or “chelating agent” means an active agent capable of removing a metal ion from a system by forming a complex so that the metal ion cannot readily participate in or catalyze chemical reactions. The inclusion of a chelating agent is especially useful for providing protection against UV radiation which can contribute to excessive scaling or skin.

Flavonoids suitable for use in the present invention are flavanones; chacones; isoflavones; coumarins; chromones; one or more dicoumarols; one or more chromanones; one or more chromanols.

A safe and effective amount of an anti-inflammatory/soothing agent may be added to the compositions of the present invention, These include Steroidal anti-inflammatory agents, including but not limited to, corticosteroids and nonsteroidal anti-inflammatory agents, and also so-called“natural” anti-inflammatory agents for example bisabolol, aloe vera, plant sterols, kola extract, chamomile, red clover extract, and compounds of the Licorice (the plant genus/species Glycvrrhiza glabra) family, including glycyrrhetic acid, glycyrrhizic acid, and derivatives thereof (e.g., salts and esters).

The compositions of the present invention may also contain a safe and effective amount of an anti-cellulite agent such as xanthine compounds (e.g., caffeine, theophylline, theobromine, and aminophylline).

The compositions of the present invention may also contain a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexyl-caine, procaine, cocaine, ketamine, pramoxine, phenol, and pharmaceutically acceptable salts thereof.

The compositions of the present invention may contain a tanning active such as dihydroxyacetone or erythrulose as an artificial tanning active.

The compositions of the present invention may contain a skin lightening agent including kojic acid, arbutin, ascorbic acid and derivatives thereof (e.g., magnesium ascorbyl phosphate or sodium ascorbyl phosphate), and extracts (e.g., mulberry extract, placenta extract).

The compositions of the present invention may contain an antimicrobial or antifungal active. Examples of antimicrobial and antifungal actives include B-lactam drugs, quinolone drugs, ciprofloxacin, norfloxacin, tetracycline, erythromycin. amikacin, 2,4,4′-trichloro-2′ hydroxy diphenyl ether, 3,4,4′-trichlorobanilide, phenoxyethanol, phenoxy propanol, phenoxyisopropanol, doxycycline, capreomycin, chlorhexidine. chlortetracycline, oxytetracycline, clindamycin, ethambutol. hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride. amanfadine sulfate, octopirox, parachlorometa xylenol, nystatin, tolnaftate, zinc pyrithione and clotrimazole.

Preferred examples of actives useful herein include those selected from salicylic acid, benzoyl peroxide, 3-hydroxy benzoic acid, glycolic acid, lactic acid, 4-hydroxy benzoic acid, acetyl salicylic acid, 2-hydroxybutanoic acid, 2-hydroxypentanoic acid. 2-hydroxyhexanoic acid, cis-retinoic acid, trans-retinoic acid, retinol, phytic acid, N-acetyl-L-cysteine, lipoic acid, azelaic acid, arachidonic acid, benzoylperoxide, tetracycline, ibuprofen, naproxen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neocycin sulfate, and mixtures thereof.

The compositions of the present invention may contain a particulate materials such as; bismuth oxychloride, iron oxide, mica, mica treated with barium sulfate and Ti02, silica, nylon, polyethylene, talc, styrene, polyproylene. ethylene/acrylic acid copolymer, sericite. aluminum oxide, silicone resin, barium sulfate, calcium carbonate, cellulose acetate, titanium dioxide, polymethyl methacrylate, and mixtures thereof.

The compositions of the present invention may contain a conditioning agent selected from humectants, moisturizers, or skin conditioners. These materials include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, glucosamine); hyaluronic acid; lactamide monoethanolamine; acetamide monoethanolamine; panthenol; allantoin; and mixtures thereof.

The compositions of the present invention can contain one or more thickening and structuring agents such as, Carboxylic Acid Polymers (Carbomers), Crosslinked Polyacrylate Polymers, Polyacrylamide Polymers, Polysaccharides such as carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof, and Gums such as acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

The cosmetic formulation containing the cereal product described herein has improved water retention, oil binding, pH buffering and moisturizing properties. The process allows the product to be certified as Natural (Ecocert), whereas traditional processes cannot make this claim.

In one embodiment the cosmetic formulation is a bath powder containing 10 to 60%, preferably about 45 w % of a cereal product described here or prepared as described here. Preferably the cereal is colloidal oatmeal (INCI: Avena sativa Kernel Meal). In one embodiment the formulation also contains oils, starch, silica and laureth-4.

In one embodiment the cosmetic formulation is a baby balm, face mask or body lotion containing 1 to 10 w-% of a cereal product described here or prepared as described here.

Also combinations of the above described cereal product and other active ingredients are included in the scope of the invention. These can be used, for example, in products for sun care, rosacea, specific anti-ageing etc.

Such combination products can comprise:
a) a safe and effective amount, preferably from 50 to 0.1%, of a cereal based active selected from the group consisting of grain meals, colloidal oatmeals, extruded colloidal oatmeals and derivatives thereof, and mixtures thereof;
b) a safe and effective amount of at least one additional active; and
c) a dermatologically acceptable carrier.

The additional active is preferably selected from the group consisting of desquamatory actives, anti-acne actives, vitamin B compounds, retinoids, peptides, hydroxy acids, anti-oxidants, radical scavengers, chelators, antiinflammatory agents, topical anesthetics, tanning actives, skin lightening agents, anti-cellulite agents, anti-wrinkle actives, flavonoids, antimicrobial actives, skin soothing agents, skin healing agents, antifungal actives, sunscreen actives, conditioning agents, structuring agents, thickening agents, herbal extracts, and mixtures thereof.

The invention is illustrated by the following non-limiting examples. It should be understood, however, that the embodiments given in the description above and in the examples are for illustrative purposes only, and that various changes and modifications are possible within the scope of the invention.

### Examples of Colloidal Oatmeal

### Example 1

### Whole Grain Oat Flour Extrusion in a Pilot Extruder

Whole grain oat flour (dry matter 87%) was extruded using a Clextral BC21. The retention time in the extruder was 31 seconds. The feed was at 4.6 kg/h and the extruder screw speed at 80 rpm. Extruder temperature was maintained along the extruder barrel at 110° C. to 120° C. The extruder did not have a nozzle plate or nozzles in order to decrease the energy consumption and torque during extrusion. The pressure in the extruder was maintained by the use of a reverse screw at the end of the barrel. The pressure generated by the reverse screw was high enough to keep the water in the liquid state without steam formation, a phenomenon which enhances the destruction of microbes.

The extrusion of whole grain oat flour without a nozzle plate and nozzles gave surprising results as shown in Table 1. The conditions in the extrusion also without added water were effective enough to kill the microbes existing in the whole grain oat flour.

TABLE 1

| | | Inlet | Outlet | | | | |
|---|---|---|---|---|---|---|---|
| | | mois- | mois- | Water | | | |
| | Temp. | ture | ture | activity | Microbes | Yeast | Moulds |
| | [° C.] | w % | w % | Aw | cfu/g | cfu/g | cfu/g |
| Flour | | 13% | — | 0.6 | 7 × 10⁵ | 5400 | 730 |
| Test 1 | 120 | 13% | 7.5% | 0.4 | <10 | <10 | <10 |
| Test 2 | 120 | 19% | 12.2% | 0.6 | <10 | <10 | <10 |
| Test 3 | 110 | 13% | 7.2% | 0.3 | <10 | <10 | <10 |
| Test 4 | 110 | 19% | 11.9% | 0.6 | <10 | <10 | <10 |

### Example 2

### Whole Grain Wheat Flour Extrusion in a Pilot Extruder

Whole grain wheat flour (dry matter 87.7 w %) with 5 w % added sunflower oil was extruded using a Clextral BC21 extruder. The retention time was 31 seconds. The feed rate was 4.6 kg/hour and the extruder screw speed at 80 rpm. Extruder temperature was maintained at 120° C. throughout the barrel. The nozzle plate and nozzles were not fitted to the extruder in order to decrease the energy consumption and torque. The pressure in the extruder was maintained by the reverse screw and was of a smaller pitch that that used in oat flour test procedure. The pressure generated by the reverse screw was high enough to keep the water in the liquid state without steam formation, a phenomenon which enhances the destruction of microbes.

The extrusion of whole grain wheat flour without a nozzle plate and nozzles gave surprising results as shown in Table 2. The conditions in the extrusion without added water were effective enough to kill the microbes existing in the whole grain wheat flour.

TABLE 2

| | | Inlet | Outlet | | | |
|---|---|---|---|---|---|---|
| | Temp. | moisture | moisture | Microbes | Yeast | Moulds |
| | [° C.] | w % | w % | cfu/g | cfu/g | cfu/g |
| Flour | | | 12.3% | 8 × 10³ | <100 | <100 |
| Test 1 | 120 | 12.3% | 8.2% | <10 | <10 | <10 |

### Example 3

### Whole Grain Oat Flour Extrusion in an Industrial Scale Extruder

Whole grain oat flour (dry matter 86.8%) was extruded using a Clextral BC72 extruder. The retention time was 25 seconds and the feed was 304 kg/hour with an extruder screw speed of 130 rpm. The extruder temperature was equalised along the barrel at 120° C. excluding the first section. The extruder did not have a nozzle plate or nozzles fitted in order to decrease the energy consumption and enhance passage through the barrel during extrusion. Pressure in the extruder was maintained by the reverse screw at the barrel exit. The pressure generated by the reverse screw was high enough to keep the water in the liquid state without steam formation, a phenomenon which enhances the destruction of microbes. The extruded products were conditioned (heater-cooler) in a belt drier and fine milled to a finished product. The microbial samples were taken after the conditioner. Analysis results are shown in Table 3.

TABLE 3

| | | Inlet | Outlet | | | |
|---|---|---|---|---|---|---|
| | Temp. | moisture | moisture | Microbes | Yeast | Moulds |
| | [° C.] | w % | w % | cfu/g | cfu/g | cfu/g |
| Flour | | 13.0% | — | 7 × 10⁵ | 5400 | 730 |
| Test 1 | 120 | 13.0% | 9.4% | <10 | <10 | <10 |

The conditions in the industrial scale extrusion without added water were effective enough to kill the microbes existing in the whole grain oat flour.

### Example 4

### Comparison of Viscosities of Extruded and Non-Extruded Colloidal Oatmeal Measured Against Shear Rate

Extruded whole grain oat flour (Example 3.) was fine milled (Görgens, Turborotor G-55) on an industrial scale in order to reach the colloidal oatmeal specification (The United States Pharmacopeial Convention USP 32-NF27). For comparison standard oat flakes were fine milled with the same mill in order to reach the same colloidal oatmeal specification. The relative viscostatic behaviours were compared to each other. The viscosity/shear rate curves of extruded and non-extruded colloidal oatmeal were measured with a Bohlin 88 viscometer with the measuring head C 25 (spindle cylinder 25 mm, outer cylinder 27.5 mm) enl. DIN 53019. Dry matter was 10 w % and temperature 20° C. The results of viscosities versus shear rate are shown in FIG. 1.

Both oatmeals had non-Newtonian viscosity behaviour, which demonstrates that they had the highest viscosities at the low shear rate and lowest viscosities at the highest shear rate. This viscosity behaviour is associated with soluble betaglucan. We confirmed that the viscosity was largely caused by betaglucan. A betaglucanase enzyme (Econase CE, AB Enzymes) was added to both oatmeal suspensions at 10 w % dry matter, which decreased the viscosity immediately to a level that corresponds to water and was not measurable with the Bohlin 88 instrument. Alpha-amylase was also added, which had no effect on the viscosities.

The viscosity of extruded colloidal oatmeal was significantly higher than the viscosity of non-extruded oatmeal. The higher viscosity of extruded version showed that it had increased dissolved beta glucan than non-extruded version. We evaluated the dissolved beta glucan concentrations of extruded and non-extruded oatmeal suspensions at 10 w % dry matter. Brix-values of centrifuged clear liquids of both samples were measured, as in this connection the Brix assay indicates an approximate concentration of dissolved solids. The Brix value of extruded version was 1.2 (1.2 w %) and non-extruded version 0.4 (0.4 w %). We know that dehulled Finnish oat varieties contain approximately 5 w % betaglucan (dry matter basis), which indicates that the highest dissolved concentration of betaglucan in oatmeal suspension at 10 w % dry matter is approximately 0.5 w %. A significant part of the difference of the dissolved solid concentrations (0.8 w %) can be attributed to the beta glucan concentration. Maunsell et al (2011) have observed the beneficial effects of betaglucan in cosmetic applications.

The viscosity difference between the extruded and non-extruded versions was highest, at the point of lowest shear rate. As result of this the viscosity measured at the lowest shear rate demonstrates the best effect of the extrusion process on maximising the availability of the beta glucan in the colloidal oatmeal. In this example the lowest shear rate was 23.3 1/s, which was achieved, when stirring rate of the spindle was 20 rpm.

### Example 5

### The Viscosity Upper Limit of 0.1 Pas Defined by USPC Monograph USP 32-NF27: Measured from Extruded and Non-Extruded Colloidal Oatmeal

The viscosities were measured according to the method defined in the United States Pharmacopeial Convention. A 25 g sample was added to 500 ml of water in small portions whilst being stirred at 1000 rpm over 1 minute. This procedure results in a flour suspension where the dry matter concentration was 4.5 w %. The starting temperature of the water was 45° C. and it was maintained at this temperature throughout the mixing stage. Stirring was continued for 5 minutes after the addition of the last portion of oatmeal. The suspension was left to stand for 90 minutes and equalise to ambient temperature. The suspension was stirred at 800 rpm for 1 minute. The viscosity was then measured using a Brookfield viscometer (DV-II+) fitted with spindle no 1 and set to 60 rpm. The spindle 1 has a cylinder 1.88 cm in diameter and 6.25 cm high attached to shaft of 0.32 cm in diameter, the distance from the top of the cylinder to the lower tip of the shaft being 0.75 cm and the immersion depth being 8.15 cm. This arrangement outlines the geometry when the viscosity is measured, and creates repeatable conditions without defining the exact shear rate. Both the extruded version and non-extruded version had lower viscosities than 0.1 Pas.

### Example 6

### Colloidal Behaviour of the Extruded and Non-Extruded Colloidal Oatmeal

The colloidal oatmeal samples were mixed to 5 w % suspension and centrifuged in a laboratory centrifuge (3000 rpm, 10 min). The tubes were then photographed. The non-extruded version is on the left and extruded version on the right. The photo is presented in FIG. 2.

The non-extruded version has clear layers and smaller layer of solids on the bottom. The extruded version has a hazy top layer and more solids on the bottom. This behaviour indicates that the extruded version has enforced colloidal behaviour.

### Example 7

### Comparison of the Particles of Extruded and Non-Extruded Colloidal Oatmeal

Extruded whole grain oat flour (Example 3) was fine milled in an industrial mill to the colloidal oatmeal specification (United States Pharmacopeial Convention USP 32-NF27). Standard oat flakes were fine milled in the same mill in order to reach the same colloidal oatmeal specification. Particles of the both colloidal oatmeals were stained with Lugol-iodine solution and the particles photographed under a microscope (FIG. 3). The extruded version is on the left and non-extruded version on the right.

The pictures show the extruded version may have more damaged particles. The most significant indication is however that the extruded material has increased blue colour and the non-extruded, a darker violet colour. The blue colour indicates that the amylose fraction is more freely available in the extruded version. Extrusion is known to increase the amylose-lipid complexes, which will extend the shelf life of the lipids and therefore would show the same effect in finished products (Asp and Björck 1998).

### Example 8

### Comparison of Oil Binding Capacities of Extruded and Non-Extruded Colloidal Oatmeal

30 grams of rapeseed oil (30 g) was added to 5 grams of the two variants of oatmeals and placed in ultracentrifuge tubes. All samples were thoroughly mixed for 45 seconds to achieve a fully homogeneous state. After allowing the samples to rest for 30 min the samples were centrifuged at 5000 rpm for 30 min. The separated oil was decanted and the precipitate weighed. The increase of the weight of the precipitate (oatmeal) was calculated. The oil binding capacity was expressed as grams of rapeseed oil bound by 100 g of oatmeal. The experiment was performed three times. Oatmeals were used in an as-is state with moisture contents of 5.4 and 7.9 w % respectively for native and extruded oatmeals. The results are shown in the table below.

TABLE 4

| | Weight | | Oil binding | Oil binding |
|---|---|---|---|---|
| | increase | Average | (g/100 g as | (g/100 g |
| Sample | (g) | (g) | is) | DM) |
| Extruded oatmeal | 3.89 | 3.9 | 78 | 85 |
| sample 1. | | | | |
| Extruded oatmeal | 3.91 | | | |
| sample 2. | | | | |
| Extruded oatmeal | 3.92 | | | |
| sample 3. | | | | |
| Native oatmeal sample 1. | 3.02 | 3.0 | 60 | 63 |
| Native oatmeal sample 2. | 2.98 | | | |
| Native oatmeal sample 3. | 3.02 | | | |

Extrusion increased the oil binding of the dry matter of oatmeal by 35% i.e. the oil binding capacity of the extruded oatmeal was significantly higher than that of the standard oatmeal.

### Examples of Cosmetic Formulations Containing Colloidal Oatmeal

### Example 9

### Soothing Natural Baby Balm (Formulation 1)

Whole grain oat flour was extruded (example 3) and fine milled (example 4) in order to reach the colloidal oatmeal specification (United States Pharmacopeial Convention USP 32-NF27). The product was tested in Soothing Natural Baby Balm (Oat Services UK, JB Code OS BB1).

| | Common/ | | | |
|---|---|---|---|---|
| Phase | Trade Name | Supplier | INCI Name | % w/w |
| A | Water | | Aqua/Water | 30.1 |
| | Rheocare XG | BASF | Xanthan Gum | 0.40 |
| | Glycerin | | Glycerin | 5.00 |
| | (vegetable) | | | |
| | Geogard Ultra | Lonza | Gluconolactone (and) | 1.00 |
| | | | Sodium Benzoate | |
| B | Sunflower Oil | A&E | Helianthus Annuus | 30.00 |
| | | Connock | Seed Oil | |
| | Castor Oil | A&E | Ricinus communis | 10.00 |
| | | Connock | (Castor) Seed Oil | |
| | Sisterna | Sisterna | Sucrose Tetrastearate | 3.50 |
| | A10E-C | | Triacetate | |
| | Illipe Butter, | Beraca | Shorea Stenoptera | 10.00 |
| | Natural | | Seed Butter | |
| | Lexgard | Inolex | Glyceryl Caprylate | 1.00 |
| | GMCY | | | |
| C | Sisterna SP70 C | Sisterna | Sucrose Stearate | 3.50 |
| | Dermofeel | Dr | Tocopherol, | 0.50 |
| | Toco 70 Non | Straetmans | Helianthus Annuus | |
| | GMO | | (Sunflower) Seed Oil | |
| D | Colloidal | Oat | Avena Sativa Kernel | 5.00 |
| | Oatmeal | Cosmetics | Meal | |

Method of Manufacture:

Phase A was heated to 50° C. and blended by stirring. Phase B was heated to 50-60° C. until it melted. Phase C was added to the heated phase B and mixed until homogeneous. Phase A was added to phase B/C with a high shear mixer. Once fully mixed, the colloidal oatmeal was added under homogenisation. Once the colloidal oatmeal was fully dispersed, and the resultant balm was poured into containers and allowed to cool.

The thick, soft balm leaves a silky but protective film on the baby's skin. The natural emulsion utilises the increased soothing benefits of colloidal oatmeal to help protect against skin issues such as diaper rash and help reduce any redness or irritation.

The product has an excellent cosmetic appearance, being a light ivory stable balm with a powdery and non-greasy after-feel on the skin.

### Example 10

### Soothing Oatmeal and Lavender Bath Powder (Formulation 2)

Whole grain oat flour was extruded (example 3) and fine milled (example 4) in order to reach colloidal oatmeal specification (United States Pharmacopeial Convention USP 32-NF27). The product was tested in a soothing oatmeal and lavender bath powder (Oat Services UK, JB Code OS BP1).

TABLE 5

| | Common/Trade | | | |
|---|---|---|---|---|
| Phase | Name | Supplier | INCI Name | % w/w |
| A | Sunflower Oil | A&E Connock | Helianthus Annuus | 20.00 |
| | | | Seed Oil | |
| | Natrasorb Bath | Akzo Nobel | Tapioca Starch | 28.00 |
| | Procol LA-4 | Protameen | Laureth-4 | 5.00 |
| | | Chemicals | | |
| | Colloidal | Oat Cosmetics | Avena Sativa Kernel | 45.00 |
| | Oatmeal | | Meal | |
| B | Lavender | S&D Aroma | Lavandula | 0.60 |
| | Essential Oil | | Angustifolia | |
| | | | (Lavender) Oil | |
| | Aerosil 200 | Evonik Industries | Silica | 1.40 |

Method of Manufacture:

The sunflower oil and Procol LA-4 of phase A were blended together. Natrasorb Bath was added and mixed thoroughly. The colloidal oatmeal was then added and mixed thoroughly. The lavender essential oil was blended with the Aerosil 200 of phase B and once homogeneous mixed into phase A. The complete powder was mixed until entirely homogenous and smooth.

This powder product is suitable for packaging in small single use sachets or “tea bags”. The colloidal powder can easily be added directly to the bath water, or the tea bag is steeped in the warm water, to provide a soothing and moisturising effect. The colloidal oatmeal gives improved release of the beneficial active components, whilst the sunflower oil is effectively emulsified into the water to provide the additional moisturising properties of bath oil without any associated greasiness. Finally the lavender oil provides a pleasant odour with an aromatherapy calming benefit.

The colloidal oatmeal helps to absorb the oils and surfactant and provide a suitably dry powder. This could also be used in the shower as a gentle body exfoliator that is rinsed off, leaving soft, soothed skin.

### Example 11

### Purifying and Moisturising Face Mask (Formulation 3)

Whole grain oat flour was extruded (example 3) and fine milled (example 4) in order to reach colloidal oatmeal specification (United States Pharmacopeial Convention USP 32-NF27). The product was tested in a purifying and moisturising face mask (Oat Services UK, JB Code OS CM2).

TABLE 6

| | Common/ | | | |
|---|---|---|---|---|
| Phase | Trade Name | Supplier | INCI Name | % w/w |
| A | Keltrol CG-SFT | CP Kelco | Xanthan Gum | 0.50 |
| | Water | | Aqua | 46.00 |
| | Glycerine (veg) | | Glycerin | 5.00 |
| | Dermosoft PEA | Dr Straetmans | Phenethyl Alcohol | 1.00 |
| | eco | | | |
| B | Pretiox AV-01- | Precolor | Titanium Dioxide | 1.00 |
| | FG | | | |
| C | Dermorganics | Dr Straetmans | Glyceryl Stearate | 6.00 |
| | GMS-SE | | SE | |
| | Sunflower Oil | A&E Connock | Helianthus Annuus | 5.00 |
| | | | Seed Oil | |
| | Lexgard GMCY | Inolex | Glyceryl Caprylate | 0.50 |
| D | Amazonian | Beraca | Kaolin | 10.00 |
| | White Clay | | | |
| | RFAWC 3900 | | | |
| | Colloidal | Oat Cosmetics | Avena Sativa Kernel | 5.00 |
| | Oatmeal | | Meal | |
| | Rose Water | Zahra | Rosa Damascena | 10.00 |
| | | Rosewater Co. | (Rose) Flower | |
| | | | Distillate | |
| E | Grain Alcohol | | Ethanol Denat. | 10.00 |

Method of Manufacture:

Xanthan gum was dispensed in the water of phase A by stirring. Phase A was heated to 75-80° C. Phase B was added and mixed until homogeneous. Phase C was heated to 75-80° C. and Phase NB added using a propeller stirrer. The resultant mixture was homogenised whilst phase D was added to the batch. The batch was then cooled to room temperature whilst under sweep agitation. Phase E was added when the temperature was below 40° C.

The creamy face mask is applied to the skin and allowed to dry before rinsing off with tepid water. The emulsion base contains both colloidal oatmeal to soothe and calm, along with clay to draw impurities out of the skin. The mask is suitable for those people who find simple clay masks too astringent and require a more hydrating and soothing option.

The colloidal oatmeal provides skin care benefits whilst not interfering with the appearance or drying of the mask and providing a soft skin feel.

### Example 12

### All Natural After-Sun Body Lotion (Formulation 4)

Whole grain oat flour was extruded (example 3) and fine milled (example 4) in order to reach colloidal oatmeal specification (United States Pharmacopeial Convention USP 32-NF27). The product was tested in all natural after-sun body lotion (Oat Services UK, JB Code OC OM 3.1).

TABLE 7

| | Common/ | | | |
|---|---|---|---|---|
| Phase | Trade Name | Supplier | INCI Name | % w/w |
| A | Water | | Aqua/Water | 74.10 |
| | Glycerin | | Glycerin | 5.00 |
| | Dermosoft 1388 | Dr Straetmans | Glycerin, Aqua, | 3.00 |
| | Eco | | Sodium Levulinate, | |
| | (preservative) | | Sodium Anisate | |
| | Dermofeel PA-3 | Dr Straetmans | Sodium Phytate, | 0.10 |
| | | | Aqua | |
| | Keltrol CG-RD | CP Kelco | Xanthan Gum | 0.20 |
| B | Symbiomuls GC | Dr Straetmans | Glyceryl Stearate | 5.00 |
| | | | Citrate, Cetearyl | |
| | | | Alcohol, Glyceryl | |
| | | | Caprylate | |
| | Apricot Kernel | Azelis | Prunus Armeniaca | 2.00 |
| | Oil | | (Apricot) Kernel Oil | |
| | Jojoba Oil, | Desert Whale | Simmondsia | 1.00 |
| | Colourless | | Chinenesis | |
| | | | (Jojoba) Seed Oil | |
| | Dermofeel | Dr Straetmans | Isoamyl Laurate | 3.00 |
| | Sensolv | | | |
| | Plantec Olive | CRM | Squalane | 1.00 |
| | Squalane | International | | |
| | Copherol | BASF | Tocopherol | 0.10 |
| | F1300 G | | | |
| | DL-Tocopheryl | DSM | Tocopheryl Acetate | 0.50 |
| | Acetate | | | |
| C | Trealix | Sinerga | Trehalose, | 2.00 |
| | | | Hydrolyzed | |
| | | | Vegetable Protein | |
| | Colloidal | Oat Cosmetics | Avena Sativa Kernel | 3.00 |
| | Oatmeal | | Meal | |

Method of Manufacture:

The xanthan gum was dispersed in the water, and when homogeneous the other ingredients of phase A were added. Phases A and B were heated separately to 78° C. Phase B was added to A using a paddle stirrer and then homogenised for 1-2 minutes. Stirring and cooling was continued and phase C was then added when the mixture was below 45° C. Stirring was continued until the mixture was cool.

This natural soft cream leaves a moisturising film on the skin. Colloidal oat flour helps to soothe the skin and provides a slightly powdery skin feel. (Kurtz and Wallo, 2007)

### Example 13

### Aftersun Lotion (Formulation 5)

| | % w/w |
|---|---|
| Aqua | to 100 |
| Hydrated silica | 5 |
| Isopropyl palmitate | 4 |
| Arachidyl propionate | 2 |
| Dimethicone | 2 |
| Glycerin | 2 |
| Steareth-21 | 1.96 |
| Steareth-2 | 1.683 |
| Cetyl alcohol | 1 |
| Tribehenin | 1 |
| Glyceryl stearate | 1 |
| Paraffinum liquidum | 0.994 |
| Panthenol | 0.75 |
| Parfum | 0.3 |
| Xanthan gum | 0.3 |
| Sodium citrate | 0.25 |
| Tocopheryl acetate | 0.2 |
| Hydroxyethylcellulose | 0.1 |
| Bisabolol | 0.095 |
| Citric acid | 0.05 |
| Preservative | q.s |
| Extruded colloidal oatmeal | 5 |

### Method

### Stage 1

The citric acid, sodium citrate and hydroxyethylcellulose are added to the water. Using a propeller stirrer, the mixture is stirred until dispersed. The xanthan gum is pre-dispersed in the glycerin and this is then added to the bulk, which is then heated to 70° C.

### Stage 2

The isopropyl palmitate, arachidyl propionate, dimethicone, steareth-21, steareth-2, cetyl alcohol, tribehenin, glyceryl stearate, paraffinum liquidum are mixed and heated to 70° C. to melt the waxes.

### Stage 3

Using a homogeniser, stage 2 is added to stage 1 and is mixed until emulsified and uniform. The emulsion is cooled to below 35° C. using stirring. Once below 35° C., the remaining materials are added, including the Extruded colloidal oatmeal. The product is made to weight using purified water, and mixed until uniform.

### Example 14

### Aftersun Lotion (Formulation 6)

| | % w/w |
|---|---|
| Aqua | to 100 |
| Hydrated silica | 5 |
| Isopropyl palmitate | 4 |
| Arachidyl propionate | 2 |
| Dimethicone | 2 |
| Glycerin | 2 |
| Steareth-21 | 1.96 |
| Steareth-2 | 1.683 |
| Cetyl alcohol | 1 |
| Tribehenin | 1 |
| Glyceryl stearate | 1 |
| Paraffinum liquidum | 0.994 |
| Panthenol | 0.75 |
| Parfum | 0.3 |
| Xanthan gum | 0.3 |
| Sodium citrate | 0.25 |
| Tocopheryl acetate | 0.2 |
| Hydroxyethylcellulose | 0.1 |
| Bisabolol | 0.095 |
| Citric acid | 0.05 |
| Preservative | q.s |
| Extruded colloidal oatmeal | 10 |

### Method

### Stage 1

The citric acid, sodium citrate and hydroxyethylcellulose are added to the water. Using a propellor stirrer, the mixture is stirred until dispersed. The xanthan gum is pre-dispersed in the glycerin and this is then added to the bulk, which is then heated to 70° C.

### Stage 2

The isopropyl palmitate, arachidyl propionate, dimethicone, steareth-21, steareth-2, cetyl alcohol, tribehenin, glyceryl stearate, paraffinum liquidum are mixed and heated to 70° C. to melt the waxes.

### Stage 3

Using a homogeniser, stage 2 is added to stage 1 and is mixed until emulsified and uniform. The emulsion is cooled to below 35° C. using stirring. Once below 35° C., the remaining materials are added, including the Extruded colloidal oatmeal. The product is made to weight using purified water, and mixed until uniform.

### Example 15

### Day Cream (Formulation 7)

| | % w/w |
|---|---|
| Aqua | to 100 |
| Butylene glycol | 5 |
| Dicaprylyl maleate | 4 |
| Paraffinum liquidum | 4 |
| Octyl methoxycinnamate | 3 |
| Petrolatum | 3 |
| Cetyl Alcohol | 2 |
| Glycerin | 2 |
| Dimethicone | 2 |
| Cetearyl alcohol | 1.6 |
| Butyl methoxydibenzoylmethane | 1 |
| Hydroxyethylcellulose | 0.4 |
| PEG-20 stearate | 0.4 |
| Polyacrylamide | 0.4 |
| Parfum | 0.3 |
| C13-14 isoparaffin | 0.215 |
| Retinyl palmitate | 0.15 |
| Tetrasodium EDTA | 0.1 |
| Citric acid | 0.08 |
| Laureth-7 | 0.055 |
| BHT | 0.0024 |
| Extruded colloidal oatmeal | 1 |
| Preservative | q.s |

### Method

### Stage 1

Tetrasodium EDTA and citric acid are added to the water using a propellor stirrer. The hydroxyethylcellulose is added and dispersed using a homogeniser. butylene glycol, glycerin and methylparaben are added and the bulk is heated to 70° C.

### Stage 2

The dicaprylyl maleate, paraffinum liquidum, octyl methoxycinnamate, petrolatum, cetyl alcohol, dimethicone, cetearyl alcohol, butyl methoxydibenzoylmethane, PEG-20 stearate, C13-14 isoparaffin, laureth-7 and BHT are mixed and heated to 70° C. to melt the waxes.

### Stage 3

Using a homogeniser, stage 2 is added to stage 1 and the bulk is mixed until emulsified and stable. The product is then cooled to below 35° C. using stirring. The remaining raw materials, including the Extruded colloidal oatmeal are added and the product is mixed using a propellor stirrer until uniform. The product is made to weight using purified water.

### Example 16

### Day Cream (Formulation 8)

| | % w/w |
|---|---|
| Aqua | to 100 |
| Butylene glycol | 5 |
| Dicaprylyl maleate | 4 |
| Paraffinum liquidum | 4 |
| Octyl methoxycinnamate | 3 |
| Petrolatum | 3 |
| Cetyl Alcohol | 2 |
| Glycerin | 2 |
| Dimethicone | 2 |
| Cetearyl alcohol | 1.6 |
| Butyl methoxydibenzoylmethane | 1 |
| Hydroxyethylcellulose | 0.4 |
| PEG-20 stearate | 0.4 |
| Polyacrylamide | 0.4 |
| Parfum | 0.3 |
| C13-14 isoparaffin | 0.215 |
| Retinyl palmitate | 0.1782 |
| Tetrasodium EDTA | 0.1 |
| Citric acid | 0.08 |
| Laureth-7 | 0.055 |
| BHT | 0.0024 |
| Extruded colloidal oatmeal | 5 |
| Preservative | q.s |

### Method

### Stage 1

Tetrasodium EDTA and citric acid are added to the water using a propellor stirrer. The hydroxyethylcellulose is added and dispersed using a homogeniser. butylene glycol, glycerin and methylparaben are added and the bulk is heated to 70° C.

### Stage 2

The dicaprylyl maleate, paraffinum liquidum, octyl methoxycinnamate, petrolatum, cetyl alcohol, dimethicone, cetearyl alcohol, butyl methoxydibenzoylmethane, PEG-20 stearate, C13-14 isoparaffin, laureth-7 and BHT are mixed and heated to 70° C. to melt the waxes.

### Stage 3

Using a homogeniser, stage 2 is added to stage 1 and the bulk is mixed until emulsified and stable. The product is then cooled to below 35° C. using stirring. The remaining raw materials, including the Extruded colloidal oatmeal are added and the product is mixed using a propellor stirrer until uniform. The product is made to weight using purified water.

### Example 17

### Sun Lotion (Formulation 9)

| | % w/w |
|---|---|
| Aqua | to 100 |
| C12-15 Alkyl Benzoate | 8 |
| Butylene glycol | 5 |
| Butyl methoxydibenzoylmethane | 2.2 |
| Dimethicone | 2 |
| Polyglyceryl-3 methylglucose distearate | 2 |
| PVP/hexadecene copolymer | 1.75 |
| Octyl methoxycinnamate | 1.7 |
| Theobroma cacao | 0.5 |
| Parfum | 0.5 |
| Tocopheryl acetate | 0.2 |
| Acrylates/vinyl isodecanoate crosspolymer | 0.15 |
| Potassium hydroxide | 0.034 |
| Tetrasodium EDTA | 0.02 |
| Preservative | q.s |
| Extruded colloidal oatmeal | 3 |

### Method

### Stage 1

The EDTA is dispersed into the water. Using a propellor stirrer, the acrylates/vinyl isodecanoate crosspolymer are added and dispersed and hydrated. Butylene glycol is added and the aqueous phase is heated to 70° C.

### Stage 2

The C12-15 alkyl benzoate, butyl methoxydibenzoylmethane, dimethicone, polyglyceryl-3 methylglucose distearate, PVP/hexadecene copolymer, octyl methoxycinnamate, theobroma cacao and tocopheryl acetate are mixed and heated to 70° C. to melt the waxes.

### Stage 3

Using a homogeniser, stage 2 is added to stage 1 and the bulk is mixed until emulsified and uniform. The emulsion is cooled to below 35° C. with stirring. The remaining materials, including the Extruded colloidal oatmeal are added and mixed. The product is made to weight using purified water and stirred until uniform.

### Example 18

### Sun Lotion (Formulation 10)

| | % w/w |
|---|---|
| Aqua | to 100 |
| C12-15 Alkyl Benzoate | 8 |
| Butylene glycol | 5 |
| Butyl methoxydibenzoylmethane | 2.2 |
| Dimethicone | 2 |
| Polyglyceryl-3 methylglucose distearate | 2 |
| PVP/hexadecene copolymer | 1.75 |
| Octyl methoxycinnamate | 1.7 |
| Theobroma cacao | 0.5 |
| Parfum | 0.5 |
| Tocopheryl acetate | 0.2 |
| Acrylates/vinyl isodecanoate crosspolymer | 0.15 |
| Potassium hydroxide | 0.034 |
| Tetrasodium EDTA | 0.02 |
| Preservative | q.s |
| Extruded colloidal oatmeal | 5 |

### Method

### Stage 1

The EDTA is dispersed into the water. Using a propellor stirrer, the acrylates/vinyl isodecanoate crosspolymer are added and dispersed and hydrated. Butylene glycol is added and the aqueous phase is heated to 70° C.

### Stage 2

The C12-15 alkyl benzoate, butyl methoxydibenzoylmethane, dimethicone, polyglyceryl-3 methylglucose distearate, PVP/hexadecene copolymer, octyl methoxycinnamate, theobroma cacao and tocopheryl acetate are mixed and heated to 70° C. to melt the waxes.

### Stage 3

Using a homogeniser, stage 2 is added to stage 1 and the bulk is mixed until emulsified and uniform. The emulsion is cooled to below 35° C. with stirring. The remaining materials, including the Extruded colloidal oatmeal are added and mixed. The product is made to weight using purified water and stirred until uniform.

### Example 19

### Sunburn Treatment (Formulation 11)

| | % w/w |
|---|---|
| Aqua | to 100 |
| Petrolatum | 3 |
| Cetyl Alcohol | 2 |
| Dimethicone | 2 |
| Glycerin | 2 |
| Ceteath-20 | 1.7 |
| Paraffinum Liquidum | 1 |
| Sodium chloride | 0.8 |
| Theobroma cacao | 0.7 |
| Glyceryl stearate | 0.5 |
| Parfum | 0.3 |
| Allantoin | 0.2 |
| Hydroxyethylcellulose | 0.1 |
| riclosan | 0.1 |
| Citric acid | 0.02 |
| Preservative | q.s |
| Extruded colloidal oatmeal | 10 |

### Method

### Stage 1

Into the water, sodium chloride and citric acid are added and dispersed. Using a propellor stirrer, hydroxyethylcellulose is added and dispersed. This phase is then heated to 70° C.

### Stage 2

The petrolatum, cetyl alcohol, dimethicone, ceteath-20, paraffinum liquidum, theobroma cacao and glyceryl stearate are mixed and heated to 70° C. to melt the waxes.

### Stage 3

Using a homogeniser, stage 2 is added to stage 1, this is mixed until emulsified and uniform. The emulsion is then cooled to below 35° C. with stirring. The remaining materials, including the Extruded colloidal oatmeal are then added and mixed. The product is made to weight using purified water and stirred until uniform.

### Example 20

### Sunburn Treatment (Formulation 12)

| | % w/w |
|---|---|
| Aqua | to 100 |
| Petrolatum | 3 |
| Cetyl Alcohol | 2 |
| Dimethicone | 2 |
| Glycerin | 2 |
| Ceteath-20 | 1.7 |
| Paraffinum Liquidum | 1 |
| Sodium chloride | 0.8 |
| Theobroma cacao | 0.7 |
| Glyceryl stearate | 0.5 |
| Parfum | 0.3 |
| Allantoin | 0.2 |
| Hydroxyethylcellulose | 0.1 |
| Triclosan | 0.1 |
| Citric acid | 0.02 |
| Preservative | q.s |
| Extruded colloidal oatmeal | 7 |

### Method

### Stage 1

Into the water, sodium chloride and citric acid are added and dispersed. Using a propellor stirrer, hydroxyethylcellulose is added and dispersed. This phase is then heated to 70° C.

### Stage 2

The petrolatum, cetyl alcohol, dimethicone, ceteath-20, paraffinum liquidum, theobroma cacao and glyceryl stearate are mixed and heated to 70° C. to melt the waxes.

### Stage 3

Using a homogeniser, stage 2 is added to stage 1, this is mixed until emulsified and uniform. The emulsion is then cooled to below 35° C. with stirring. The remaining materials, including the Extruded colloidal oatmeal are then added and mixed. The product is made to weight using purified water and stirred until uniform.

### Example 21

### Eye Cream (Formulation 13)

| | % w/w |
|---|---|
| Aqua | to 100 |
| Butylene glycol | 6 |
| Paraffinum liquidum | 5 |
| Octyl methoxycinnamate | 4 |
| Dimethicone | 2 |
| Petrolutum | 2 |
| Cetearyl octanoate | 1.8 |
| Cetearyl alcohol | 1.6 |
| Glyceryl stearate | 1.5 |
| Cetyl alcohol | 1 |
| Prunus dulcis | 1 |
| Glycerin | 0.57 |
| Hydrogenated vegetable glycerides citrate | 0.5 |
| Tocopheryl acetate | 0.5 |
| Bisabolol | 0.475 |
| Panthenol | 0.45 |
| Sodium phosphate | 0.42 |
| PEG-20 stearate | 0.4 |
| Isopropyl myristate | 0.2 |
| Carbomer | 0.15 |
| PEG-12 isostearate | 0.125 |
| Allantoin | 0.1 |
| Tetrasodium EDTA | 0.1 |
| Lactic acid | 0.088 |
| Disodium phophate | 0.083 |
| Potassium hydroxide | 0.051 |
| Extruded colloidal oatmeal | 1 |
| Preservative | q.s |

### Method

### Stage 1

Into the water, citric acid, EDTA, sodium phosphate, disodium phosphate and lactic acid are added and dispersed. Using a homogeniser, carbomer is added and hydrated. The aqueous phase is then heated to 70° C.

### Stage 2

The paraffinum liquidum, octyl methoxycinnamate, dimethicone, petrolatum, cetearyl octanoate, cetearyl alcohol, glyceryl stearate, cetyl alcohol, hydrogenated vegetable glycerides citrate, tocopheryl acetate, PEG-20 stearate, isopropyl myristate and PEG-12 isostearate are mixed and heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 22

### Eye Cream (Formulation 14)

| | % w/w |
|---|---|
| Aqua | to 100 |
| Butylene glycol | 6 |
| Paraffinum liquidum | 5 |
| Octyl methoxycinnamate | 4 |
| Dimethicone | 2 |
| Petrolutum | 2 |
| Cetearyl octanoate | 1.8 |
| Cetearyl alcohol | 1.6 |
| Glyceryl stearate | 1.5 |
| Cetyl alcohol | 1 |
| Prunus dulcis | 1 |
| Glycerin | 0.57 |
| Hydrogenated vegetable glycerides citrate | 0.5 |
| Tocopheryl acetate | 0.5 |
| Bisabolol | 0.475 |
| Panthenol | 0.45 |
| Sodium phosphate | 0.42 |
| PEG-20 stearate | 0.4 |
| Isopropyl myristate | 0.2 |
| Carbomer | 0.15 |
| PEG-12 isostearate | 0.125 |
| Allantoin | 0.1 |
| Tetrasodium EDTA | 0.1 |
| Lactic acid | 0.088 |
| Disodium phophate | 0.083 |
| Potassium hydroxide | 0.051 |
| Extruded colloidal oatmeal | 4 |
| Preservative | q.s |

### Method

### Stage 1

Into the water, citric acid, EDTA, sodium phosphate, disodium phosphate and lactic acid are added and dispersed. Using a homogeniser, carbomer is added and hydrated. The aqueous phase is then heated to 70° C.

### Stage 2

The paraffinum liquidum, octyl methoxycinnamate, dimethicone, petrolatum, cetearyl octanoate, cetearyl alcohol, glyceryl stearate, cetyl alcohol, hydrogenated vegetable glycerides citrate, tocopheryl acetate, PEG-20 stearate, isopropyl myristate and PEG-12 isostearate are mixed and heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 23

### Eye Gel (Formulation 15)

| | % W/W |
|---|---|
| Aqua | to 100 |
| PVP/VA copolymer | 2 |
| Propylene glycol | 2 |
| Carbomer | 1 |
| PEG-40 hydrogenated castor oil | 1 |
| Panthenol | 1 |
| Sodium hydroxide | 0.3 |
| Phenoxyethanol | 0.2 |
| Tetrasodium EDTA | 0.1 |
| Extruded colloidal oatmeal | 0.5 |

### Method

### Stage 1

The EDTA, Methyldibromo glutaronitrile, PVP/VA copolymer and Carbomer were added to the water and mixed using a homogeniser to ensure that the polymers were hydrated.

### Stage 2

With continued homogenising, the Cystine hydroxypropyl polysiloxane was added and mixed into the product.

### Stage 3

The remaining materials, including the Extruded colloidal oatmeal were added individually and mixed using a prop. Strirrer until the product was homogenous.

### Example 24

### Refreshing Cream (Formulation 16)

| | % w/w |
|---|---|
| Aqua | to 100 |
| Butylene glycol | 7.5 |
| Silica | 7.2 |
| Arabinogalactan | 5.35 |
| Dimethicone | 5.35 |
| Petrolatum | 5.35 |
| Hydrated silica | 3.75 |
| Steareth-2 | 2.7 |
| Prunus dulcis | 2.7 |
| Steareth-21 | 0.9 |
| PVP/hexadecene copolymer | 0.8 |
| Carbomer | 0.32 |
| Sodium PCA | 0.2 |
| Parfum | 0.2 |
| Hydroxyethylcellulose | 0.16 |
| Potassium hydroxide | 0.1 |
| Propylene glycol | 0.1 |
| Extruded colloidal oatmeal | 1 |
| Preservative | q.s |

### Method

### Stage 1

Into the water, the carbomer is added and hydrated using a homogeniser. The aqueous phase is then heated to 70° C.

### Stage 2

The silica, arabinogalactan, PVP/hexadecene copolymer, dimethicone, petrolatum, hydrated silica, steareth-2 and steareth-21 are mixed and heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 25

### Skin Protection Lotion (Formulation 17)

| | % w/w |
|---|---|
| Aqua | to 100 |
| Dimethicone | 5 |
| Glycerin | 3 |
| Kaolin | 3 |
| Dicaprylyl maleate | 2.5 |
| Isopropyl myristate | 2.5 |
| Stearate-2 | 2 |
| Octyl methoxycinnamate | 1 |
| Steareth-21 | 1 |
| Cetyl alcohol | 0.75 |
| Butyl methoxydibenzoylmethane | 0.5 |
| Propylene glycol | 0.5 |
| Hydroxyethylcellulose | 0.4 |
| Xanthan gum | 0.24 |
| Serica | 0.1 |
| Sodium C8-16 isoalkylsuccinyl lactoglobulin sulfonate | 0.1 |
| Tetrasodium EDTA | 0.1 |
| Citric acid | 0.05 |
| Extruded colloidal oatmeal | 1 |
| Preservative | q.s |

### Method

### Stage 1

Into the water, the citric acid and EDTA are added and dispersed. The hydroxyethylcellulose is added and hydrated using a propellor stirrer. Xanthan gum is pre-dispersed in glycerin and added to the bulk. This is stirred until uniform. The aqueous phase is then heated to 70° C.

### Stage 2

The dimethicone, dicaprylyl maleate, isopropyl myristate, stearate-2, octyl methoxycinnamate, steareth-21, cetyl alcohol and butyl methoxydibenzoylmethane are mixed and heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 26

### Night Cream (Formulation 18)

| | % w/w |
|---|---|
| Aqua | to 100 |
| Glycerin | 5 |
| Paraffinum liquidum | 4.5 |
| Dicaprylyl maleate | 3 |
| Dimethicone | 3 |
| Petrolatum | 3 |
| Paraffin | 2.9 |
| Cetyl alcohol | 2 |
| Steareth-2 | 2 |
| Glyceryl stearate | 1.5 |
| Butyrospermum parkii | 1.5 |
| Steareth-21 | 1 |
| Mannitol | 1 |
| Cera microcristallina | 0.262 |
| Buxus chinensis | 0.5 |
| Propylene glycol | 0.48 |
| Parfum | 0.4 |
| Borago officinalis | 0.3 |
| Hydroxyethylcellulose | 0.3 |
| Lactis proteinum | 0.3 |
| Xanthan gum | 0.25 |
| Alcohol denat. | 0.08 |
| Sodium citrate | 0.08 |
| Lecithin | 0.075 |
| BHT | 0.05 |
| Faex | 0.04 |
| Phospholipids | 0.03 |
| Citric acid | 0.025 |
| Extruded colloidal oatmeal | 5 |
| Preservative | q.s |

### Method

### Stage 1

Into the water, the citric acid and sodium citrate are added and dispersed. The hydroxyethylcellulose is added and hydrated using a propellor stirrer. Xanthan gum is pre-dispersed in glycerin and added to the bulk. This is stirred until uniform. The aqueous phase is then heated to 70° C.

### Stage 2

The paraffinum liquidum, dicaprylyl maleate, dimethicone, petrolatum, paraffin, cetyl alcohol, steareth-2, glyceryl stearate, steareth-21, cera microcristallina and BHT are mixed and heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 27

### Sun Lotion for Sensitive Skin (Formulation 19)

| | % w/w |
|---|---|
| Aqua | to 100 |
| C12-15 alkyl benzoate | 12 |
| Butylene glycol | 5 |
| Octyl methoxycinnamate | 3.8 |
| Butyl methoxydibenzoylmethane | 3 |
| Dimethicone | 2 |
| Polyglyceryl-3 methylglucose distearate | 2 |
| PVP/hexadecene copolymer | 1.75 |
| C18-36 acid glycol ester | 1.5 |
| Polysorbate 60 | 0.5 |
| Titanium dioxide | 0.3 |
| Tocopheryl acetate | 0.2 |
| Acrylates/vinyl isodecanoate crosspolymer | 0.14 |
| Potassium hydroxide | 0.035 |
| Tetrasodium EDTA | 0.02 |
| Preservative | q.s |
| Extruded colloidal oatmeal | 5 |

### Method

### Stage 1

Into the water, citric acid is added and dispersed. The acrlyates/vinyl isodecanoate crosspolymer are added and dispersed using a propellor stirrer. The aqueous phase is then heated to 70° C.

### Stage 2

The C12-15 alkyl benzoate, PVP/hexadecene copolymer, octyl methoxycinnamate, butyl methoxydibenzoylmethane, dimethicone, polyglyceryl-3 methylglucose distearate, C18-36 acid glycol ester, polysorbate 60, titanium dioxide and tocopheryl acetate are mixed and heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 28

### Sun Lotion for Sensitive Skin (Formulation 20)

| | % w/w |
|---|---|
| Aqua | to 100 |
| C12-15 alkyl benzoate | 12 |
| Butylene glycol | 5 |
| Octyl methoxycinnamate | 3.8 |
| Butyl methoxydibenzoylmethane | 3 |
| Dimethicone | 2 |
| Polyglyceryl-3 methylglucose distearate | 2 |
| PVP/hexadecene copolymer | 1.75 |
| C18-36 acid glycol ester | 1.5 |
| Polysorbate 60 | 0.5 |
| Titanium dioxide | 0.3 |
| Tocopheryl acetate | 0.2 |
| Acrylates/vinyl isodecanoate crosspolymer | 0.14 |
| Potassium hydroxide | 0.035 |
| Tetrasodium EDTA | 0.02 |
| Preservative | q.s |
| Extruded colloidal oatmeal | 10 |

### Method

### Stage 1

Into the water, citric acid is added and dispersed. The acrylates/vinyl isodecanoate crosspolymer are added and dispersed using a propellor stirrer. The aqueous phase is then heated to 70° C.

### Stage 2

The C12-15 alkyl benzoate, PVP/hexadecene copolymer, octyl methoxycinnamate, butyl methoxydibenzoylmethane, dimethicone, polyglyceryl-3 methylglucose distearate, C18-36 acid glycol ester, polysorbate 60, titanium dioxide and tocopheryl acetate are heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 29

### Sun Cream for Sensitive Skin (Formulation 21)

| | % w/w |
|---|---|
| Aqua | to 100 |
| Octyl stearate | 13.5 |
| Zinc oxide | 13.5 |
| Isopropyl myristate | 5 |
| Butylene glycol | 3 |
| Isohexadecane | 3 |
| Titanium dioxide | 2 |
| Polyglyceryl-3 oleate | 1.75 |
| Cetyl dimethicone copolyol | 1.35 |
| Magnesium sulfate | 0.75 |
| Sodium chloride | 0.75 |
| Aluminium stearate | 0.18 |
| Alumina | 0.15 |
| Lecithin | 0.13 |
| Isopropyl palmitate | 0.05 |
| Extruded colloidal oatmeal | 1 |

### Method

### Stage 1

Into the water, magnesium sulfate, sodium chloride and butylene glycol are added and dispersed. The aqueous phase is then heated to 70° C.

### Stage 2

The octyl stearate, isopropyl myristate, isohexadecane, titanium dioxide, polyglyceryl-3 oleate, cetyl dimethicone copolyol, aluminium stearate, lecithin and isopropyl palmitate are mixed and heated to 70° C. to melt the waxes.

### Stage 3

Using a propellor stirrer, stage 2 is added to stage 1. Once uniform, the emulsion is transferred to a homogeniser and mixed to generate the viscosity. The emulsion is then cooled to below 35° C. using stirring. The remaining materials, including the Extruded colloidal oatmeal are then added and mixed. The product is then made to weight using purified water and is stirred until uniform.

### Example 30

### Sun Cream for Sensitive Skin (Formulation 22)

| | % w/w |
|---|---|
| Aqua | to 100 |
| Octyl stearate | 13.5 |
| Zinc oxide | 13.5 |
| Isopropyl myristate | 5 |
| Butylene glycol | 3 |
| Isohexadecane | 3 |
| Titanium dioxide | 2 |
| Polyglyceryl-3 oleate | 1.75 |
| Cetyl dimethicone copolyol | 1.35 |
| Magnesium sulfate | 0.75 |
| Sodium chloride | 0.75 |
| Aluminium stearate | 0.18 |
| Alumina | 0.15 |
| Lecithin | 0.13 |
| Isopropyl palmitate | 0.05 |
| Extruded colloidal oatmeal | 10 |

### Method

### Stage 1

Into the water, magnesium sulfate, sodium chloride and butylene glycol are added and dispersed. The aqueous phase is then heated to 70° C.

### Stage 2

The octyl stearate, isopropyl myristate, isohexadecane, titanium dioxide, polyglyceryl-3 oleate, cetyl dimethicone copolyol, aluminium stearate, lecithin and isopropyl palmitate are mixed and heated to 70° C. to melt the waxes.

### Stage 3

Using a propellor stirrer, stage 2 is added to stage 1. Once uniform, the emulsion is transferred to a homogeniser and mixed to generate the viscosity. The emulsion is then cooled to below 35° C. using stirring. The remaining materials, including the Extruded colloidal oatmeal are then added and mixed. The product is then made to weight using purified water and is stirred until uniform.

### Example 31

### Anti-Ageing Foundation (Formulation 23)

| | % w/w |
|---|---|
| Aqua | to 100 |
| Butylene glycol | 9.8 |
| Cetearyl isononanoate | 4.9 |
| Dimethicone | 3.2 |
| Glycerin | 1.96 |
| Silica | 1.9 |
| Caprylic/capric triglyceride | 1.67 |
| Paraffinum liquidum | 1.67 |
| Petrolatum | 1.67 |
| Hydrogenated coco-glycerides | 1.67 |
| Cetearyl octanoate | 1.5 |
| Cetearyl alcohol | 1.35 |
| Octyl methoxycinnamate | 1.28 |
| Talc | 1 |
| Glyceryl stearate | 0.95 |
| PEG-100 stearate | 0.9 |
| Butyl methoxydibenzoylmethane | 0.6 |
| Saccharide isomerate | 0.54 |
| Lactic acid | 0.45 |
| Sodium polyacrylate | 0.45 |
| Boron nitride | 0.42 |
| Sodium PCA | 0.4 |
| Borago officinalis | 0.4 |
| Tocopheryl acetate | 0.4 |
| PVP/hexadecene copolymer | 0.4 |
| PEG-20 stearate | 0.33 |
| Glycolic acid | 0.2 |
| Sodium stearoyl lactylate | 0.2 |
| Isopropyl myristate | 0.17 |
| Polyaminopropyl biguanide | 0.16 |
| Tetrasodium EDTA | 0.1 |
| Xanthan gum | 0.1 |
| Citric acid | 0.06 |
| Alcohol denat. | 0.04 |
| Lecithin | 0.037 |
| Preservative | q.s |
| Extruded colloidal oatmeal | 1 |

### Method

### Stage 1

Into the water, citric acid, EDTA and lactic acid are added and dispersed. Xanthan gum is pre-dispersed in butylene glycol and is added to the bulk. The aqueous phase is then heated to 70° C.

### Stage 2

The cetearyl isononanoate, dimethicone, silica, PVP/hexadecene copolymer, caprylic/capric triglyceride, paraffinum liquidum, petrolatum, hydrogenated coco-glycerides, cetearyl octanoate, cetearyl alcohol, octyl methoxycinnamate, talc, glyceryl stearate, PEG-100 stearate, butyl methoxydibenzoylmethane, borago officinalis, tocopheryl acetate, sodium stearoyl lactylate, isopropyl myristate and lecithinoil phase are mixed and heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 32

### Anti-Ageing Foundation (Formulation 24)

| | % w/w |
|---|---|
| Aqua | to 100 |
| Butylene glycol | 9.8 |
| Cetearyl isononanoate | 4.9 |
| Dimethicone | 3.2 |
| Glycerin | 1.96 |
| Silica | 1.9 |
| Caprylic/capric triglyceride | 1.67 |
| Paraffinum liquidum | 1.67 |
| Petrolatum | 1.67 |
| Hydrogenated coco-glycerides | 1.67 |
| Cetearyl octanoate | 1.5 |
| Cetearyl alcohol | 1.35 |
| Octyl methoxycinnamate | 1.28 |
| Talc | 1 |
| Glyceryl stearate | 0.95 |
| PEG-100 stearate | 0.9 |
| Butyl methoxydibenzoylmethane | 0.6 |
| Saccharide isomerate | 0.54 |
| Lactic acid | 0.45 |
| Sodium polyacrylate | 0.45 |
| Boron nitride | 0.42 |
| Iron Oxide | 3 |
| Titanium Dioxide | 1 |
| Sodium PCA | 0.4 |
| Borago officinalis | 0.4 |
| Tocopheryl acetate | 0.4 |
| PVP/hexadecene copolymer | 0.4 |
| PEG-20 stearate | 0.33 |
| Glycolic acid | 0.2 |
| Sodium stearoyl lactylate | 0.2 |
| Isopropyl myristate | 0.17 |
| Polyaminopropyl biguanide | 0.16 |
| Tetrasodium EDTA | 0.1 |
| Xanthan gum | 0.1 |
| Citric acid | 0.06 |
| Alcohol denat. | 0.04 |
| Lecithin | 0.037 |
| Preservative | q.s |
| Extruded colloidal oatmeal | 10 |

### Method

### Stage 1

Into the water, citric acid, EDTA and Lactic acid are added and dispersed. Xanthan gum is pre-dispersed in butylene glycol and is added to the bulk. The aqueous phase is then heated to 70° C.

### Stage 2

The cetearyl isononanoate, dimethicone, Silica, PVP/hexadecene copolymer, caprylic/capric triglyceride, paraffinum liquidum, petrolatum, hydrogenated coco-glycerides, cetearyl octanoate, cetearyl alcohol, octyl methoxycinnamate, talc, glyceryl stearate, PEG-100 stearate, butyl methoxydibenzoylmethane, borago officinalis, tocopheryl acetate, sodium stearoyl lactylate, isopropyl myristate and lecithinoil phase are mixed and heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 33

### Sun Spray—SPF15 (Formulation 25)

| | % w/w |
|---|---|
| Aqua | to 100 |
| Dicaprylyl maleate | 12 |
| Butylene glycol | 5 |
| Octyl methoxycinnamate | 4 |
| Butyl methoxydibenzoylmethane | 3.5 |
| Dimethicone | 3 |
| Polyglyceryl-3 methylglucose distearate | 3 |
| Acrylates/octylacrylamide copolymer | 2 |
| C18-36 acid glycol ester | 1.5 |
| Triethanolamine | 0.5 |
| Tocopheryl acetate | 0.2 |
| Acrylates/vinyl isodecanoate crosspolymer | 0.05 |
| Tetrasodium EDTA | 0.02 |
| Potassium hydroxide | 0.015 |
| Preservative | q.s |
| Extruded colloidal oatmeal | 1 |

### Method

### Stage 1

Into the water, EDTA is added and dispersed. Acrylates/vinyl isodecanoate crosspolymer are added and dispersed using a propellor stirrer. Butylene glycol is added and dispersed. The aqueous phase is then heated to 70° C.

### Stage 2

The dicaprylyl maleate, Acrylates/octylacrylamide copolymer, octyl methoxycinnamate, butyl methoxydibenzoylmethane, dimethicone, polyglyceryl-3 methylglucose, C18-36 acid glycol ester and tocopheryl acetate are mixed and heated to 80° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 34

### Sun Spray (Formulation 26)

| | % w/w |
|---|---|
| Aqua | to 100 |
| Dicaprylyl maleate | 12 |
| Butylene glycol | 5 |
| Octyl methoxycinnamate | 4 |
| Butyl methoxydibenzoylmethane | 3.5 |
| Dimethicone | 3 |
| Polyglyceryl-3 methylglucose distearate | 3 |
| Acrylates/octylacrylamide copolymer | 2 |
| C18-36 acid glycol ester | 1.5 |
| Triethanolamine | 0.5 |
| Tocopheryl acetate | 0.2 |
| Acrylates/vinyl isodecanoate crosspolymer | 0.05 |
| Tetrasodium EDTA | 0.02 |
| Potassium hydroxide | 0.015 |
| Preservative | q.s |
| Extruded colloidal oatmeal | 0.5 |

### Method

### Stage 1

Into the water, EDTA is added and dispersed. Acrylates/vinyl isodecanoate crosspolymer are added and dispersed using a propellor stirrer. Butylene glycol is added and dispersed. The aqueous phase is then heated to 70° C.

### Stage 2

The dicaprylyl maleate, Acrylates/octylacrylamide copolymer, octyl methoxycinnamate, butyl methoxydibenzoylmethane, dimethicone, polyglyceryl-3 methylglucose, C18-36 acid glycol ester and tocopheryl acetate are mixed and heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 35

### Toner & Cleanser 2 In 1 (Formulation 27)

| | % w/w |
|---|---|
| Alcohol denat. | 48 |
| Aqua | to 100 |
| PEG-8 | 6 |
| Glycerin | 2 |
| Propylene glycol | 0.5 |
| Sodium C8-16 isoalkylsuccinyl | 0.02 |
| lactoglobulin sulfonate | |
| Laminaria saccharina | 0.01 |
| Hamamelis virginiana | 0.006 |
| Citrullus vulgaris | 0.001 |
| Preservative | q.s |
| Extruded colloidal oatmeal | 2 |

### Method

### Stage 1

Into the water, alcohol denat. Is added and dispersed until uniform. Using a propellor stirrer, all materials including the Extruded colloidal oatmeal, are slowly added and stirred until uniform. The product is made to weight using purified water and stirred until uniform.

### Example 36

### Toner & Cleanser 2 In 1 (Formulation 28)

| | % w/w |
|---|---|
| Alcohol denat. | 48 |
| Aqua | to 100 |
| PEG-8 | 6 |
| Glycerin | 2 |
| Propylene glycol | 0.5 |
| Sodium C8-16 isoalkylsuccinyl | 0.02 |
| lactoglobulin sulfonate | |
| Laminaria saccharina | 0.01 |
| Hamamelis virginiana | 0.006 |
| Citrullus vulgaris | 0.001 |
| Preservative | q.s |
| Extruded colloidal oatmeal | 5 |

### Method

### Stage 1

Into the water, alcohol denat. Is added and dispersed until uniform. Using a propellor stirrer, all materials including the Extruded colloidal oatmeal, are slowly added and stirred until uniform. The product is made to weight using purified water and stirred until uniform.

### Example 37

### Skin pH Balancing Toner (Formulation 29)

| | % w/w |
|---|---|
| Aqua | to 100 |
| Alcohol denat. | 7.9 |
| Butylene glycol | 2 |
| Dimethicone copolyol | 1.5 |
| Sodium lactate | 0.6 |
| Glycerin | 0.5 |
| Allantoin | 0.1 |
| Propylene glycol | 0.1 |
| Lactic acid | 0.002 |
| Preservative | q.s |
| Extruded colloidal oatmeal | 1 |

### Method

### Stage 1

Into the water, lactic acid and alcohol denat are separately added and dispersed until uniform. Using a propellor stirrer, all materials including the Extruded colloidal oatmeal, are slowly added and stirred until uniform. The product is made to weight using purified water and stirred until uniform.

### Example 38

### Skin pH Balancing Toner (Formulation 30)

| | % w/w |
|---|---|
| Aqua | to 100 |
| Alcohol denat. | 7.9 |
| Butylene glycol | 2 |
| Dimethicone copolyol | 1.5 |
| Sodium lactate | 0.6 |
| Glycerin | 0.5 |
| Allantoin | 0.1 |
| Propylene glycol | 0.1 |
| Lactic acid | 0.002 |
| Preservative | q.s |
| Extruded colloidal oatmeal | 10 |

### Method

### Stage 1

Into the water, lactic acid and alcohol denat are separately added and dispersed until uniform. Using a propellor stirrer, all materials including the Extruded colloidal oatmeal, are slowly added and stirred until uniform. The product is made to weight using purified water and stirred until uniform.

### Example 39

### pH Balanced Cleansing Lotion (Formulation 31)

| | % w/w |
|---|---|
| Aqua | to 100 |
| Paraffinum liquidum | 14 |
| Isopropyl palmitate | 7 |
| Glyceryl stearate | 2.5 |
| PEG-100 stearate | 2.5 |
| Butylene glycol | 2 |
| Hydrogenated vegetable glycerides citrate | 2 |
| Polysorbate 60 | 0.5 |
| Sorbitan stearate | 0.5 |
| Persea gratissima | 0.3 |
| Prunus persica | 0.3 |
| Propylene glycol | 0.3 |
| Acrylates/C10-30 alkyl acrylate crosspolymer | 0.12 |
| Potassium hydroxide | 0.05 |
| Tetrasodium EDTA | 0.02 |
| Medicago sativa | 0.0045 |
| Preservative | q.s |
| Extruded colloidal oatmeal | 1 |

### Method

### Stage 1

Into the water, EDTA is added and dispersed. Butylene glycol is then added and dispersed. The aqueous phase is then heated to 70° C.

### Stage 2

The paraffinum liquidum, isopropyl palmitate, glyceryl stearate, PEG-100 stearate, hydrogenated vegetable glycerides citrate, polysorbate 60 and sorbitan stearate are mixed and heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 40

### pH Balanced Cleansing Lotion (Formulation 32)

| | % w/w |
|---|---|
| Aqua | to 100 |
| Paraffinum liquidum | 14 |
| Isopropyl palmitate | 7 |
| Glyceryl stearate | 2.5 |
| PEG-100 stearate | 2.5 |
| Butylene glycol | 2 |
| Hydrogenated vegetable glycerides citrate | 2 |
| Polysorbate 60 | 0.5 |
| Sorbitan stearate | 0.5 |
| Persea gratissima | 0.3 |
| Prunus persica | 0.3 |
| Propylene glycol | 0.3 |
| Acrylates/C10-30 alkyl acrylate crosspolymer | 0.12 |
| Potassium hydroxide | 0.05 |
| Tetrasodium EDTA | 0.02 |
| Medicago sativa | 0.0045 |
| Preservative | q.s |
| Extruded colloidal oatmeal | 10 |

### Method

### Stage 1

Into the water, EDTA is added and dispersed. Butylene glycol is then added and dispersed. The aqueous phase is then heated to 70° C.

### Stage 2

The paraffinum liquidum, isopropyl palmitate, glyceryl stearate, PEG-100 stearate, hydrogenated vegetable glycerides citrate, polysorbate 60 and sorbitan stearate are mixed and heated to 70° C. to melt the waxes.

### Stage 3

Using a homogeniser, stage 2 is added to stage 1 and this is mixed until emulsified and uniform. The emulsion is then cooled to below 35° C. using stirring. The remaining materials, including the Extruded colloidal oatmeal are then added and mixed. The product is then made to weight using purified water and is stirred until uniform.

### Example 41

### Lipstick (Formulation 33)

| | % w/w |
|---|---|
| 1. RICINUS COMMUNIS | 20 |
| Octyldodecanol | 15 |
| Pentaerythrityl tetracaprylate/caprate | 14 |
| Mica | 10 |
| Bis-diglyceryl caprylate/caprate/isostearate/ | 7.5 |
| Stearate/hydroxystearate adipate | |
| Paraffin | 5 |
| Cera microcristallina | 5 |
| Propylene glycol | 2 |
| Hydrogenated castor oil | 2 |
| Candelilla cera | 1 |
| Carnauba | 1 |
| Synthetic wax | 1 |
| Butyrospermum parkii | 1 |
| Titanium dioxide | 0.5 |
| Tocopheryl acetate | 0.2 |
| Polyquaternium-37 | 0.2 |
| Pigments and dyes | q.s |
| Extruded colloidal oatmeal | 1 |

### Method

### Stage 1

The Extruded colloidal oatmeal is pre-dispersed in propylene glycol, with stirring.

### Stage 2

The remaining materials are mixed in a vessel and heated to 85° C. until melted and uniform. The product is cooled and the Extruded colloidal oatmeal pre-mix is added below 70° C. The product poured into a suitable container and allowed to cool to room temperature to set.

### Example 42

### Lipstick (Formulation 34)

| | % w/w |
|---|---|
| 2. RICINUS COMMUNIS | 20 |
| Octyldodecanol | 15 |
| Pentaerythrityl tetracaprylate/caprate | 14 |
| Mica | 10 |
| Bis-diglyceryl caprylate/caprate/isostearate/ | 7.5 |
| Stearate/hydroxystearate adipate | |
| Paraffin | 5 |
| Cera microcristallina | 5 |
| Propylene glycol | 2 |
| Hydrogenated castor oil | 2 |
| 2.1 Candelilla cera | 1 |
| Carnauba | 1 |
| Synthetic wax | 1 |
| Butyrospermum parkii | 1 |
| Titanium dioxide | 0.5 |
| Tocopheryl acetate | 0.2 |
| Polyquaternium-37 | 0.2 |
| Red colour | q.s |
| Extruded colloidal oatmeal | 10 |

### Method

### Stage 1

The Extruded colloidal oatmeal is pre-dispersed in propylene glycol, with stirring.

### Stage 2

The remaining materials are mixed in a vessel and heated to 85° C. until melted and uniform. The product is cooled and the Extruded colloidal oatmeal pre-mix is added below 70° C. The product poured into a suitable container and allowed to cool to room temperature to set.

### Example 43

### Hair Conditioner (Formulation 35)

| | % W/W |
|---|---|
| Aqua | to 100 |
| Cetyl alcohol | 3 |
| Cetrimonium chloride | 0.8 |
| Hydroxyethylcellulose | 0.6 |
| Propylene glycol | 0.5 |
| Panthenol | 0.5 |
| Parfum | 0.3 |
| Benzophenone-4 | 0.2 |
| Sodium chloride | 0.1 |
| Wheat amino acids | 0.14 |
| Citric acid | 0.02 |
| Tetrasodium EDTA | 0.02 |
| Extruded colloidal oatmeal | 1 |

### Method

### Stage 1

The EDTA and Hydroxyethylcellulose were added to the water and mixed using a homogeniser to hydrate the polymer. Citric acid, Benzophenone and Cetrimonium chloride were added. This was then heated to 70 C.

### Stage 2

Cetyl alcohol was heated to 70 C. in a separate vessel.

### Stage 3

The melted Cetyl alcohol was then added to stage 1 using a homogeniser.

### Stage 4

The mixture was then cooled to below 40 C using a prop. Stirrer. The remaining materials including the Extruded colloidal oatmeal were then added and the product was made to weight with purified water.

### Example 44

### Intensive Conditioner (Formulation 36)

| | % W/W |
|---|---|
| Aqua | to 100 |
| Cetearyl alcohol | 4.5 |
| Arachidyl propionate | 2 |
| Dimethicone | 2 |
| Panthenol | 0.75 |
| Stearamidopropyl dimethylamine | 1.5 |
| Hydroxyethylcellulose | 0.5 |
| Amodimethicone | 0.7 |
| Citric acid | 0.5 |
| Cetrimonium chloride | 0.4 |
| PEG-20 stearate | 0.4 |
| Parfum | 0.3 |
| Propylene glycol | 0.3 |
| Benzophenone-4 | 0.2 |
| Sodium chloride | 0.15 |
| Wheat amino acids | 0.15 |
| Polyquaternium-39 | 0.1 |
| Extruded colloidal oatmeal | 1 |

### Method

### Stage 1

The EDTA and HEC were added to the water and mixed using a homogeniser to hydrate the polymer.

### Stage 2

The citric acid and cetrimonium chloride were added and mixed using a prop. Stirrer.

The mixture was then heated to 70 C.

### Stage 3

In a separate vessel, the waxes, dimethicone and BHT were mixed and heated to 70 C. until melted and uniform.

### Stage 4

Stage 3 was added to stage 2 and this was mixed until uniform. The mixture was then cooled to below 40 C with stirring.

### Stage 5

The remaining materials including the Extruded colloidal oatmeal were then added and the product was made to weight using purified water.

### Example 45

### Leave-in Conditioner (Formulation 37)

| | % W/W |
|---|---|
| Aqua | to 100 |
| PEG-40 hydrogenated castor oil | 2 |
| Dipropylene glycol | 1 |
| Phenoxyethanol | 0.8 |
| Parfum | 0.3 |
| Panthenol | 0.4 |
| Propylene glycol | 0.25 |
| Methylparaben | 0.2 |
| Benzophenone-4 | 0.2 |
| Extruded colloidal oatmeal | 1 |

### Method

### Stage 1

The Polyquaternium-10 was added to the water and hydrated using a prop. stirrer.

### Stage 2

The Methylparaben was pre-dispersed in Dipropylene glycol, gently heated to melt and then added to stage 1.

### Stage 3

The remaining materials including the Extruded colloidal oatmeal were then added and the product was mixed and made to weight with purified water.

### Example 46

### Gentle Shampoo (Formulation 38)

| | % W/W |
|---|---|
| Aqua | to 100 |
| Sodium laureth sulfate | 8 |
| Cocamidopropyl betaine | 3 |
| Sodium chloride | 1.8 |
| Cocamide DEA | 1.6 |
| PEG-6 cocamide | 1 |
| Parfum | 0.5 |
| Panthenol | 0.4 |
| Propylene glycol | 0.3 |
| Benzophenone-4 | 0.2 |
| Glycerin | 0.2 |
| Phenoxyethanol | 0.1 |
| Extruded colloidal oatmeal | 1 |

### Method

### Stage 1

To the water, EDTA, Sodium chloride, Citric acid and Benzophenone-4 were added. This was followed by the addition of Sodium laureth sulfate, Methyldibromo glutaronitrile, wheat amino acids and the Extruded colloidal oatmeal.

### Stage 2

PEG-6 cocamide and Cocamide DEA were heated gently until liquified. The parfume was added and mixed. This was then added to the product.

### Stage 3

The Cocamidopropyl betaine and remaining materials, including the Extruded colloidal oatmeal were then added and mixed. The product was made to weight using purified water.

### Example 47

### Intensive Conditioner (Formulation 39)

| | % W/W |
|---|---|
| Aqua | to 100 |
| Sodium laureth sulfate | 8.5 |
| Cocamidopropyl betaine | 23 |
| Cocamide DEA | 2.2 |
| Panthenol | 1 |
| Sodium chloride | 0.8 |
| Laureth-3 | 0.5 |
| Parfum | 0.3 |
| Propylene glycol | 0.5 |
| Benzophenone-4 | 0.2 |
| Glycerin | 0.5 |
| Phenoxyethanol | 0.5 |
| Extruded colloidal oatmeal | 3 |

### Example 40

### Anti-Dandruff Shampoo (Formulation 40)

| | % W/W |
|---|---|
| Aqua | to 100 |
| Sodium laureth sulfate | 6 |
| Disodium laureth sulfosuccinate | 4 |
| Laureth-3 | 3 |
| Cocamidopropyl betaine | 2.5 |
| Sodium chloride | 2 |
| Dipropylene glycol | 1 |
| Parfum | 0.5 |
| Piroctone olamine | 0.5 |
| Panthenol | 0.4 |
| Propylene glycol | 0.3 |
| Disodium phosphate | 0.25 |
| Benzophenone-4 | 0.2 |
| Wheat amino acids | 0.15 |
| Extruded colloidal oatmeal | 2 |

### Method

### Stage 1

EDTA, Citric acid and Benzophenone-4 were added and mixed into the water. Sodium laureth sulfate, Disodium laureth sulfosuccinate and Dipropylene glycol were then added.

### Stage 2

Disodium phosphate, wheat amino acids and the Extruded colloidal oatmeal were added and the product was stirred until uniform.

### Stage 3

The Piroctone olamine was dispersed in the parfum and added to the Laureth-3. This mixture was added to the bulk and stirred.

### Stage 4

The remaining materials were then added and the product was made to weight with purified water.

### Example 49

### Anti-Chlorine Shampoo (Formulation 41)

| | % W/W |
|---|---|
| Aqua | to 100 |
| Sodium laureth sulfate | 8 |
| Cocamidopropyl betaine | 3 |
| Sodium chloride | 1.6 |
| Laureth-3 | 1 |
| Phenoxyethanol | 0.8 |
| Parfum | 0.5 |
| Disodium phosphate | 0.4 |
| Panthenol | 0.4 |
| Propylene glycol | 0.3 |
| Methylparaben | 0.2 |
| Benzophenone-4 | 0.2 |
| Wheat amino acids | 0.14 |
| Propylparaben | 0.1 |
| Sodium thiosulfate | 0.1 |
| Extruded colloidal oatmeal | 1 |

### Stage 1

To the water, the following materials were added and mixed; Benzophenone, Sodium chloride, Sodium phosphate, Disodium phosphate, EDTA.

### Stage 2

Sodium laureth sulfate, phenoxyethanol, Panthenol, Wheat amino acids and the Extruded colloidal oatmeal were then added and stirred.

### Stage 3

The preservatives were pre-mixed in the Laureth-3 and heated slightly to melt the powders. This was added to the product.

### Stage 4

The remaining materials were added and the product was made to weight using purified water.

### Example 50

### Hair Gel (Formulation 42)

| | % W/W |
|---|---|
| Aqua | to 100 |
| Cyclomethicone | 7 |
| Dimethiconol | 1 |
| Phenoxyethanol | 0.8 |
| Propylene glycol | 0.8 |
| Panthenol | 0.7 |
| Carbomer | 0.7 |
| Aminomethyl propanol | 0.4 |
| Benzophenone-4 | 0.2 |
| Parfum | 0.2 |
| Extruded colloidal oatmeal | 1 |

### Method

### Stage 1

To the water; EDTA and Benzophenone-4 were added using an homogeniser.

### Stage 2

The carbomer was added and hydrated with continued homogenising.

### Stage 3

The Phenoxyethanol, Cyclomethicone, Dimethiconol, Propylene glycol and Panthenol were then added and mixed until homogenous.

### Stage 4

The remaining materials including the Extruded colloidal oatmeal were added and the bulk was homogenised until uniform.

### Stage 5

The product was made to weight using purified water.

### Example 51

### Hair Putty (Formulation 43)

| | % W/W |
|---|---|
| Aqua | to 100 |
| Cetearyl alcohol | 11 |
| Lanolin | 7 |
| PVP | 6 |
| Paraffin | 6 |
| PVP/VA copolymer | 6 |
| Carnauba | 3 |
| Petrolatum | 2 |
| Polyquaternium-11 | 2 |
| PEG-20 stearate | 2 |
| Paraffinum liquidum | 1 |
| Propylene glycol | 0.8 |
| Phenoxyethanol | 0.6 |
| Dimethicone | 0.5 |
| Panthenol | 0.4 |
| Cetrimonium chloride | 0.35 |
| Dimethicone propyl PG-betaine | 0.25 |
| Benzophenone-4 | 0.2 |
| Methylparaben | 0.12 |
| Extruded colloidal oatmeal | 3 |

### Method

### Stage 1

To the water, the PVP/VA copolymer, PVP and Benzophenone-4 were added and stirred until homogenous. This was then heated to 70 C.

### Stage 2

In a separate vessel, the waxes were mixed and heated to 70 C. until all materials had melted.

### Stage 3

The hot waxes were then added to stage 1 and mixed using a prop. Stirrer until homogenous. The mixture was then cooled to below 60 C.

### Stage 4

The remaining materials, including the Extruded colloidal oatmeal were then added and the product was stirred until uniform.

### Stage 5

The product was made to weight using purified water.

### Example 52

### Hydrating Conditioner (Formulation 44)

| | % W/W |
|---|---|
| Aqua | to 100 |
| Cetyl alcohol | 4 |
| Dimethicone | 2 |
| Hydroxyethylcellulose | 0.8 |
| Cetrimonium chloride | 0.8 |
| Panthenol | 0.75 |
| Propylene glycol | 0.6 |
| Parfum | 0.3 |
| Benzophenone-4 | 0.2 |
| Amodimethicone | 0.15 |
| Dimethicone propyl PG-betaine | 0.15 |
| Sodium chloride | 0.15 |
| Extruded colloidal oatmeal | 1 |

### Method

### Stage 1

To the water, EDTA and Hydroxyethylcellulose were added using homogenising to hydrate the polymer.

### Stage 2

The benzophenone-4 and Laureth-3 were then added and the bulk was heated to 70 C.

### Stage 3

In a separate vessel, the Cetyl alcohol was heated to 70 C. until melted.

### Stage 4

Using an homogeniser, the Cetyl alcohol was added to the bulk and mixed until uniform.

### Stage 5

The product was cooled and the remaining materials, including the Extruded colloidal oatmeal were then added and mixed.

### Stage 6

The product was made to weight using purified water.

### Example 53

### Spray-On Gel (Formulation 45)

| | % W/W |
|---|---|
| Phase 1 | |
| Aqua | to 100 |
| PVP/VA copolymer | 5 |
| Isopropyl alcohol | 2.5 |
| Propylene glycol | 2.3 |
| Glycerin | 2 |
| Panthenol | 0.4 |
| Benzophenone-4 | 0.2 |
| Extruded colloidal oatmeal | 1 |
| Phase 2 | |
| PEG-40 hydrogenated castor oil | 1 |
| Parfum | 0.5 |
| Phase 3 | |
| Alcohol denat. | 45 |
| Dimethicone copolyol | 1 |

### Method

### Stage 1

The materials in phase 1 were mixed until uniform using a prop. Stirrer. Stage 2

The materials in phase 2 were pre-mixed and added to phase 1.

### Stage 3

The materials in phase 3 were mixed and added to the bulk.

### Stage 4

The product was made to weight using purified water.

### Example 54

### Shampoo for Dry Scalps (Formulation 46)

| | % W/W |
|---|---|
| Aqua | to 100 |
| Sodium laureth sulfate | 7 |
| Sodium chloride | 2.5 |
| Cocamidopropyl betaine | 2 |
| Laureth-3 | 1 |
| Panthenol | 0.5 |
| Propylene glycol | 0.5 |
| Piroctone olamine | 0.25 |
| Benzophenone-4 | 0.2 |
| Extruded colloidal oatmeal | 1 |

### Method

### Stage 1

To the water, EDTA, Citric acid, Benzophenone-4 and Sodium chloride were added and mixed using a prop. Stirrer until all materials were dissolved and uniform.

### Stage 2

The Sodium laureth sulfate and Piroctone Olamine were then added and stirred until homogenous.

### Stage 3

The remaining materials, including the Extruded colloidal oatmeal were then added and the product was stirred until uniform and homogenous.

### Stage 4

The product was made to weight with purified water.

### Example 55

### Deep Cleansing Shampoo (Formulation 47)

| | % W/W |
|---|---|
| Aqua | to 100 |
| Sodium laureth sulfate | 15 |
| Sodium chloride | 2.8 |
| Cocamidopropyl betaine | 1.5 |
| PEG-6 cocamide | 1 |
| Parfum | 0.15 |
| Panthenol | 0.4 |
| Propylene glycol | 0.3 |
| Benzophenone-4 | 0.2 |
| Phenoxyethanol | 0.1 |
| Extruded colloidal oatmeal | 1 |

### Method

### Stage 1

To the water, Citric acid, EDTA and Sodium chloride were added and dissolved.

### Stage 2

The Benzophenone-4, Sodium laureth sulfate, Cocamidopropyl betaine, Panthenol, Methydibromo glutaronitrile, Wheat amino acids and the Extruded colloidal oatmeal were then added and mixed until the product was uniform, using a prop. Stirrer.

### Stage 3

The parfum was pre-dispersed in the PEG-6 cocamide and then added to the bulk.

### Stage 4

The product was made to weight using purified water.

### Example 56

### Hydrating Shampoo (Formulation 48)

| | % W/W |
|---|---|
| Aqua | to 100 |
| Sodium laureth sulfate | 8 |
| Cocamidopropyl betaine | 3 |
| Cocamide DEA | 1.7 |
| Panthenol | 1.2 |
| Sodium chloride | 1.5 |
| Laureth-3 | 1.2 |
| Parfum | 0.5 |
| Propylene glycol | 0.5 |
| Polyquaternium-10 | 0.4 |
| Glycerin | 0.2 |
| Extruded colloidal oatmeal | 1 |

### Method

### Stage 1

To the water, the EDTA and Polyquaternium-10 were added and the polymer was hydrated using an homogeniser.

### Stage 2

The Citric acid, Sodium chloride and Benzophenone-4 were added and stirred until uniform.

### Stage 3

The remaining materials, including the Extruded colloidal oatmeal were added individually and the product was mixed using a prop. Stirrer until homogenous.

### Stage 4

The product was made to weight using purified water.

### Example 57

### Extra Hold Hair Gel (Formulation 49)

| | % W/W |
|---|---|
| Aqua | to 100 |
| PVP/VA copolymer | 2 |
| Propylene glycol | 1.3 |
| Carbomer | 1 |
| PEG-40 hydrogenated castor oil | 1 |
| Panthenol | 0.6 |
| Sodium hydroxide | 0.3 |
| Parfum | 0.3 |
| Phenoxyethanol | 0.2 |
| Tetrasodium EDTA | 0.1 |
| Mica | 0.1 |
| Cystine hydroxypropyl polysiloxane | 0.1 |
| Extruded colloidal oatmeal | 1 |

### Method

### Stage 1

The EDTA, Methyldibromo glutaronitrile, PVP/VA copolymer and Carbomer were added to the water and mixed using a homogeniser to ensure that the polymers were hydrated.

### Stage 2

With continued homogenising, the Cystine hydroxypropyl polysiloxane was added and mixed into the product.

### Stage 3

The remaining materials, including the Extruded colloidal oatmeal were added individually and mixed using a prop. Stirrer until the product was homogenous.

### Examples of Colour Cosmetic Formulations Containing Colloidal Oatmeal

### Example 58

### Pressed Powder (Colour Cosmetics Formulation 1)

| | % w/w |
|---|---|
| Phase A | |
| Talc and methicone 1 | 61.25 |
| Nylon 12 2 | 15.00 |
| Extruded Colloidal Oatmeal | 5.00 |
| Tocopherol/glycerine-vitamin E | 1.40 |
| Iron oxide red 3 | 2.35 |
| Iron oxide yellow 3 | 2.35 |
| Iron oxide black 3 | 0.60 |
| Titanium oxide 4 | 4.00 |
| Preservative/Fragrance | q.s. |
| Phase B | |
| BC 2161 Dimethicone and | 4.50 |
| Trimethylsilyloxysilicate 5 | |
| BC 2231 | 3.50 |
| Cyclopentasiloxane and | |
| Dimethiconol 5 | |

### Method

### Stage 1

Combine phase A using a ribbon blender and micropulverise.

### Stage 2

Combine phase B, separately, and heat the mixture to 50° C.

### Stage 3

Spray phase B into the phase A mixture while blending.

### Stage 4

Micropulverise until homogeneous and press at 1200-1500 PSI.

### Example 59

### Loose Bronze Powder (Colour Cosmetics Formulation 2)

| Material | % w/w |
|---|---|
| Part 1 | |
| Kobo Mica I 27 | 18.15 |
| Mica powder Y 3000 | 15 |
| Polymethyl methacrylate BPA 500 | 5 |
| Talc | to 100 |
| Extruded colloidal Oatmeal | 7 |
| Iron Oxides (C.I. 77492) (And) Isopropyl Titanium | 0.2 |
| Triisostearate (And) Triethoxysilylethyl | |
| Polydimethylsiloxyethyl Dimethicone | |
| Iron Oxides (C.I. 77491) (And) Isopropyl Titanium | 0.1 |
| Triisostearate (And) Triethoxysilylethyl | |
| Polydimethylsiloxyethyl Dimethicone | |
| Iron Oxides (C.I. 77499) (And) Triethoxysilylethyl | 0.05 |
| Polydimethylsiloxyethyl Dimethicone (And) | |
| Isopropyl Titanium Triisostearate | |
| Preservatives | 0.25 |
| Part 2 | |
| Dimethicone | 4.00 |
| dl-alpha Tocopherol Acetate | 1.00 |
| Part 3 Pearls | |
| Mixed colours pearls Mica (And) Iron Oxides (C.I. 77491) | 30 |
| Mixed colours pearls Mica (And) Titanium Dioxide (And) | 3.5 |
| Iron Oxides (C.I. 77491) | |
| Mixed colours pearls Titanium Dioxide (And) Mica (And) | 2.5 |
| Iron Oxides (C.I. 77491) | |

### Method

### Stage 1

Weigh and add Part 1 raw materials to the Waring Blender under a fume hood.

Mix for 2-3 minutes.

### Stage 2

Pre-mix Part 2.

### Stage 3

Add Part 2 slowly to Part 1 under a fume hood. Mix for 2 minutes.

### Stage 4

Add the Pearls and mix for 1-2 minutes.

### Example 60

### Creme-Powder Blush (Colour Cosmetics Formulation 3)

| | % w/w |
|---|---|
| Part 1 | |
| SERICITE GMS-4C Mica | 17 |
| RBTD-I2 Titanium Dioxide (And) Isopropyl Titanium | 10 |
| Triisostearate | |
| Extruded Colloidal Oatmeal | 3 |
| SPC/MST-547-I2 Polymethylsilsesquioxane (And) | 6 |
| Ethylene/Methacrylate Copolymer (And) Isopropyl | |
| Titanium Triisostearate | |
| BYO-I2 Iron Oxides (C.I. 77492) (And) Isopropyl | 0.33 |
| Titanium Triisostearate | |
| BRO-I2 Iron Oxides (C.I. 77491) (And) Isopropyl | 0.33 |
| Titanium Triisostearate | |
| BBO-I2 Iron Oxides (C.I. 77499) (And) Isopropyl | 0.1 |
| Titanium Triisostearate | |
| Preservatives | 0.2 |
| Part 2 | |
| Wickenol 155 Ethylhexyl Palmitate | 32.55 |
| Squalane NF Squalane | 12.00 |
| Microcrystalline Wax SP-89 Microcrystalline Wax | 6.00 |
| Lameform TGI Polyglycerol-3 Diisostearate | 5.50 |
| Mineral Oil Carnation Mineral Oil | 3.00 |
| Softisan ® 100 Hydrogenated | 2.00 |
| Coco-Glycerides | |
| Carnauba Wax SP 63 Copernicia Cerifera | 2.00 |
| (Carnauba) Wax | |

### Method

### Stage 1

Blend Part 1 and pass through a micronizer until the color is fully dispersed.

### Stage 2

Heat Part 2 with stirring to 91-93° C. Maintain temperature for 30 minutes.

### Stage 3

Add Part 1 to Part 2 and mix until homogeneous. Stir and cool to 88° C. Add Part 3.

### Stage 4

Continue to mix until uniform while maintaining temperature. Fill at 85° C.

### Example 61

### Non-Transfer Lipstick (Colour Cosmetics Formulation 4)

| | % w/w |
|---|---|
| Part 1 | |
| KOBOGUARD ® HRPC Hydrogenated | 20.00 |
| Polycyclopentadiene (And) Polyethylene (And) | |
| Copernicia Cerifera (Carnauba) Wax (And) | |
| Tocopherol | |
| Extruded colloidal oatmeal | 5 |
| INBP75ER Iron Oxides (C.I. 77491) (And) Isononyl | 7.25 |
| Isononanoate (And) Isopropyl Myristate (And) Stearalkonium | |
| Hectorite (And) Polyhydroxystearic Acid (And) Isopropyl | |
| Titanium Triisostearate (And) Propylene Carbonate | |
| Candelilla Wax SP 75 Euphorbia Cerifera (Candelilla) Wax | 7.00 |
| SERICITE GMS-4C Mica | 5.00 |
| Ozokerite Wax White SP 1020P Ozokerite | 4.50. |
| Microcrystalline Wax SP-89 Microcrystalline Wax | 2.00 |
| Part 2 | |
| Permethyl 99AD Isododecane | 49.25 |

### Method

*Use explosion-proof mixers and equipment during batching process*

### Stage 1

Combine Part 1 and heat to 90° C. Mix well under propeller until color is fully dispersed.

### Stage 2

Cool to 80° C. and add Part 2.

### Stage 3

Pour into molds.

What is claimed is:

1. A cereal product having a solubility corresponding to a value of over 0.4 w % dissolved solids evaluated in 10 w % water suspension according to the Brix method.

2. The product of claim 1 that is natural.

3. The product of claim 1 having a microbial count less than 1000 cfu/g.

4. The product of claim 1 having a microbial count less than 100 cfu/g.

5. The product of claim 1 wherein the cereal product is of colloidal nature.

6. The product of claim 1 having a viscosity of at least 0.150 Pas measured at 20° C. from a water suspension containing 10 w % of dry matter at a shear rate in the range of 20 to 40 1/s.

7. The product of claim 1 having improved oil absorption properties compared to non-extruded flour product.

8. The product of claim 1 wherein the cereal constituent is oats.

9. A method for producing a cereal product comprising the steps of extruding the cereal flour and fine grinding, wherein the conditions in extrusion chamber are controlled by
a) maintaining the moisture content below 17 w %; or
b) optionally adding oil(s) to the feed at 1 to 6 w %; or
c) lowering pressure of the extrusion chamber by 20 w %; or
d) any combination of steps (a) to (c).

10. The method of claim 9 wherein the conditions in extrusion chamber are controlled by:
a) maintaining the moisture content below 15 w %; or
b) optionally adding oil(s) to the feed at 2 to 4 w %; or
c) lowering pressure of the extrusion chamber by 40 w %; or
d) any combination of steps (a) to (c).

11. The method of claim 9, wherein the conditions in extrusion chamber are controlled by
a) maintaining the moisture content below 13 w %; or
b) optionally adding oil(s) to the feed to about 3 w %; or
c) lowering pressure of the extrusion chamber by 60 w % compared to conventional pressures; or
d) any combination of steps (a) to (c).

12. The method of claim 9 wherein the pressure is lowered by the use of a reverse screw.

13. The use of the product according to claim 1 in a cosmetic formulation.

14. The use of the product according to claim 1 in a food product.

15. The use of the product according to claim 1 as a coating barrier in protective products.

16. A cosmetic product containing a cereal product according to claim 1.

17. The cosmetic product of claim 16, wherein the product comprises:
a) a safe and effective amount, preferably from 50 to 0.1%, of a cereal based active selected from the group consisting of grain meals, colloidal oatmeals, extruded colloidal oatmeals and derivatives thereof, and mixtures thereof;
b) a safe and effective amount of at least one additional active; and
c) a dermatologically acceptable carrier.

18. The cosmetic composition according to claim 17, wherein the additional active is selected from the group consisting of desquamatory actives, anti-acne actives, vitamin B compounds, retinoids, peptides, hydroxy acids, anti-oxidants, radical scavengers, chelators, antiinflammatory agents, topical anesthetics, tanning actives, skin lightening agents, anti-cellulite agents, anti-wrinkle actives, flavonoids, antimicrobial actives, skin soothing agents, skin healing agents, antifungal actives, sunscreen actives, conditioning agents, structuring agents, thickening agents, herbal extracts, and mixtures thereof.

19. A cosmetic product according to claim 16 being a bath powder containing 10 to 60 w % of a cereal product.

20. A cosmetic product of claim 16, being baby balm, face mask or body lotion containing 1 to 10 w % of a cereal product.

21. The use of the product obtained according to the method of claim 9 in a cosmetic formulation.

22. The use of the product obtained according to the method of claim 9 in a food product.

23. The use of the product according to claim 9 obtained as a coating barrier in protective products.

24. A cosmetic product containing a cereal product according to the method of claim 9.

25. A cosmetic product being a bath powder containing 10 to 60 w % of a cereal product according to claim 1.

26. A cosmetic product being a bath powder containing 10 to 60 w % of a cereal product obtained according to the method of claim 9.

27. A cosmetic product, being baby balm, face mask or body lotion containing 1 to 10 w % of a cereal product according to claim 1.

28. A cosmetic product, being baby balm, face mask or body lotion containing 1 to 10 w % of a cereal product obtained according to the method of claim 9.
